(19) United States
(12) Patent Application Publication
(10) Pub. No.: US 2015/0340612 A1
(43) Pub. Date: Nov. 26, 2015

(54) UV EMITTERS COMPRISING A MULTIPLE BOND

(71) Applicant: MERCK PATENT GMBH, Darmstadt (DE)

(72) Inventors: JUNYOU PAN, Frankfurt Am Main (DE); Martin ENGEL, Darmstadt (DE); Herwig BUCHHOLZ, Frankfurt Am Main (DE)

(73) Assignee: MERCK PATENT GMBH, Darmstadt (DE)

(21) Appl. No.: 14/759,047

(22) PCT Filed: Dec. 9, 2013
(86) PCT No.: PCT/EP2013/003714

§ 371 Date: Jul. 2, 2015

(30) Foreign Application Priority Data
Jan. 3, 2013 (EP) 13000011.0

(51) Int. Cl. H01L 51/00; A61N 5/06; C09K 11/06

(52) U.S. Cl. CPC H01L 51/005; C09K 11/06; A61N 5/06; H01L 51/5004

(57) ABSTRACT

The invention relates to organic electroluminescent devices comprising organic compounds with a double or triple bond, to which at least one aromatic ring is bonded, as emitter compounds. The invention also relates to possible uses of said devices.

### CROSS-REFERENCE TO RELATED APPLICATIONS

This application is a national stage application (under 35 U.S.C. §371) of PCT/EP2013/003714, filed Dec. 9, 2013, which claims benefit of European Application No. 13000011.0, filed Jan. 3, 2013, both of which are incorporated herein by reference in their entirety.

The present invention relates to organic electroluminescent devices which comprise organic compounds having a double or triple bond, to which at least one aromatic ring is bonded, as emitter compounds and to possible uses thereof.

The structure of organic light-emitting diodes (OLEDs) in which organic semiconductors are employed as functional materials is described, for example, in U.S. Pat. No. 4,539,507, U.S. Pat. No. 5,151,629, EP 0 676 461 and WO 98/27136. The emitting materials employed here, besides fluorescent emitters, are increasingly organometallic complexes which exhibit phosphorescence (M. A. Baldo et al., Appl. Phys. Lett. 1999, 75, 4-6). OLEDs represent a very highly promising technology for display screen and lighting applications. To this end, OLEDs are necessary which emit light in the visible region of the spectrum, i.e. typically red, green and blue light.

Furthermore, there are many applications which require light or radiation having even shorter wavelengths. Thus, for example, in the area of life science and medicine, wavelengths of in the range from 280 to 400 nm are necessary for so-called “cell imaging” or for biosensors. Furthermore, in the electronics industry, wavelengths from 300 to 400 nm are required for so-called “solid-state lighting” and from 300 to 365 nm, for example, for the curing of polymers and printing ink. Also of major importance are phototherapeutic applications in the medical or cosmetics sector. Many undesired skin changes and skin diseases can be treated by means of phototherapy. Wavelengths in the region of ultraviolet (UV) radiation are often required for this purpose. An example thereof is the treatment of the skin of psoriatic patients, for which purpose a radiation source which emits UV radiation of a wavelength of 311 nm is typically employed.

Mercury, deuterium, excimer and xenon lamps are typical, conventional UV radiation sources. However, they are unwieldy and some contain toxic substances which may cause soiling and may represent health risks. The conventional lamps therefore have disadvantages regarding safety, usability, handling ability and portability, which in turn results in limited possible applications. In addition, UV-LEDs are also commercially available. However, most of these LEDs are either at the research stage, only emit radiation having a wavelength greater than 365 nm or are very expensive. In addition, LEDs have the disadvantage that they are point emitters, which require relatively thick and rigid devices. Another class of radiation sources or light sources are the organic electroluminescent devices (for example OLEDs or OLECs—organic light-emitting electrochemical cells). In contrast to the other light and radiation sources, these are area emitters. Furthermore, the organic electroluminescent devices allow the production of flexible equipment, such as displays, lighting devices and irradiation devices. These devices are also particularly suitable for many applications owing to their efficiency and the simple and space-saving structure.

However, only very little is known to date about organic electroluminescent devices which emit radiation in the UV region. The emission of most organic electroluminescent devices is usually limited to wavelengths greater than 350 nm. In addition, the performance data of these devices are very poor.

Chao et al. report (Adv. Mater. 17[8], 992-996. 2005.) on UV-OLEDs based on fluorene polymers having an electroluminescence emission wavelength greater than 360 nm;

Wong et al. report (Org. Lett. 7[23], 5131-5134. 2005) on UV-OLEDs based on spirobifluorene polymers having an electroluminescence emission wavelength at 360 nm or greater;

Zhou et al. report (Macromolecules 2007, 40 (9), 3015-3020) on UV-OLEDs comprising emitting polymers based on fluorene and tetraphenylsilane derivatives having an electroluminescence emission wavelength at 350 nm;

Shinar et al. report (Applied Surface Science 2007, 254 (3), 749-756) on UV-OLEDs using Bu-PBD as emitter having an electroluminescence emission wavelength of 350 nm.

Burrows reports (Applied Physics Letters 2006, 88 (18), 183503) on an OLED comprising 4,4′-bis(diphenylphosphine oxide) biphenyl as emitter. The device emits at 337 nm.

Sharma et al., report (Applied Physics Letters 2006, 88 (14), 143511-143513) on a UV-OLED which emits at 357 nm. The emitter used is based on polysilane.

For the above-mentioned reasons, it would be desirable to develop organic electroluminescent devices which emit radiation in the UV region, in particular in the lower UV-A region (315 to 380 nm) and in the UV-B region (280 to 315 nm). A particular challenge here is the provision of suitable organic emitter materials and the provision of organic electroluminescent devices comprising these emitters.

The object of the present invention is therefore to overcome the said disadvantages of the prior art by the provision of organic electroluminescent devices having the best-possible physical properties which exhibit an emission in the UV region.

Surprisingly, it has been found that certain compounds, described in greater detail below, achieve these objects and result in organic electroluminescent devices having unexpectedly good properties. The present invention therefore relates to organic electroluminescent devices which comprise compounds of this type.

In an embodiment, the present invention provides an organic electroluminescent device which comprises at least two electrodes and at least one emitting layer (emitter layer or emission layer) between the electrodes. The emitting layer comprises at least one compound of the following formula (1) or (2):

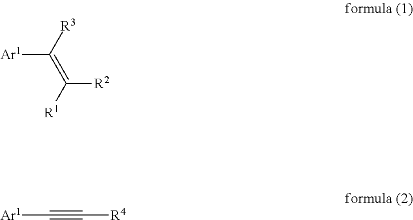

where the symbols used have the following meanings:
Ar¹ is an aromatic or heteroaromatic ring having 5 or 6 ring atoms, which may be substituted by one or more radicals R⁶, or a bicyclic condensed aromatic or heteroaromatic ring system having 10 to 12 ring atoms, which may be substituted by one or more radicals R⁶;
R¹, R², R³ and R⁴
   are, independently of one another on each occurrence, H, D, F, Ar², N(R⁵)₂, CN, Si(R⁵)₃, B(OR⁵)₂, P(R⁵)₂, S(═O)R⁵, a straight-chain alkyl, alkoxy or thioalkoxy group having 1 to 40 C atoms or a branched or cyclic alkyl, alkoxy or thioalkoxy group having 3 to 40 C atoms, each of which may be substituted by one or more radicals R⁶, where one or more non-adjacent CH₂ groups may be replaced by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵ and where one or more H atoms may be replaced by D, F, Cl, or CN, or an aryloxy, arylalkoxy or heteroaryloxy group having 5 to 60 aromatic ring atoms, which may in each case be substituted by one or more radicals R⁶, or a combination of two or more of these groups; where, in addition, two or more of the radicals R¹, R², R³ and R⁴ may form a mono- or polycyclic ring system, which may be substituted by one or more radicals R⁸, with one another; where R¹ may also be a divalent group W, which is linked to Ar¹ of the formula (1), and where, if R² is equal to Ar², R³ may also be a divalent group W which is linked to Ar²;
Ar² has the same meaning as Ar¹;
W is, identically or differently on each occurrence, a non-aromatic bridge between the double or triple bond of the formula (1) or (2) and Ar¹ or Ar² and contains O, S, Se, N, Si, B, P and/or at least one group C(R⁷)₂;
R⁵ is, identically or differently on each occurrence, H, D, F, OH, N(R⁷)₂, CN, Si(R⁷)₃, B(OR⁷)₂, P(═O)(R⁷)₂, P(R⁷)₂, S(═O)R⁷, a straight-chain alkyl, alkoxy or thioalkoxy group having 1 to 40 C atoms or a branched or cyclic alkyl, alkoxy or thioalkoxy group having 3 to 40 C atoms, each of which may be substituted by one or more radicals R⁸, where one or more non-adjacent CH₂ groups may be replaced by R⁷C═CR⁷, C≡C, Si(R⁷)₂, Ge(R⁷)₂, Sn(R⁷)₂, C═O, C═S, C═Se, C═NR⁷, P(═O)(R⁷), SO, SO₂, NR⁷, O, S or CONR⁷ and where one or more H atoms may be replaced by D, F, Cl or CN, or an aromatic ring system having 6 to 18 aromatic ring atoms or a heteroaromatic ring system having 5 to 18 aromatic ring atoms, each of which may be substituted by one or more radicals R⁸, or a combination of two or more of these groups; two or more adjacent radicals R⁷ or R⁸ may form a mono- or polycyclic aliphatic ring system with one another here;
R⁶ has the same meaning as R⁵, but cannot be equal to H;
R⁷ is, identically or differently on each occurrence, H, D, F, a straight-chain alkyl, alkoxy or thioalkoxy group having 1 to 40 C atoms or a branched or cyclic alkyl, alkoxy or thioalkoxy group having 3 to 40 C atoms, an aromatic ring system having 6 to 18 aromatic ring atoms or a heteroaromatic ring system having 5 to 18 aromatic ring atoms, in which, in addition, one or more H atoms may be replaced by F; two or more substituents R⁷ may also form a mono- or polycyclic aliphatic ring system with one another here;
R⁸ has the same meaning as R⁷, but cannot be equal to H;
   with the proviso that the compound of the formula (1) or (2) does not contain any through-conjugated structure whose number of π electrons is more than 18, preferably not more than 14 π electrons and even more preferably not more than 12 π electrons.

In a further preferred embodiment of the electroluminescent device according to the invention, Ar¹ or Ar² is, independently of one another on each occurrence, a compound of the following formula (3) or (4):

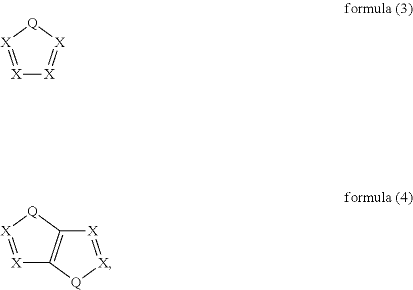

where Ar¹ or Ar² can be bonded to the double bond or triple bond of the formula (1) or (2) at any desired and chemically possible site of the compound of the formula (3) or (4); and
where the symbols used have the following meanings:
Q is on each occurrence, identically or differently, X═X, NR⁵, O, S or Se;
X is on each occurrence, identically or differently, CR⁵ or N;
R⁵ has the same meaning as defined above.

### BRIEF DESCRIPTION OF THE FIGURES

FIGS. 1 to 4, 10 and 11 show the electroluminescence (EL) and photoluminescence spectra (PL) of devices according to the invention comprising emitters E1 to E4, E11 and E12.

FIGS. 5 to 9, 12 and 13 show the photoluminescence spectra of devices according to the invention comprising emitters E6 to E10, E13 and E14.

FIG. 14 shows the combined phospholuminescence spectrum of devices according to the invention comprising emitters E2 to E4.

In a further preferred embodiment, R¹ in the electroluminescent device according to the invention is equal to H, F or a group W which is linked to Ar¹.

In a further preferred embodiment, R² in the electroluminescent device according to the invention is equal to Ar², an arylalkyl group which is substituted by a radical R⁶ in the alkyl unit, or, together with R³, forms a divalent unit —CH₂—(C(R⁷)₂)ₕ—CH₂—, where h is equal to 1, 2 or 3, preferably 2. It is preferred here that only one of the radicals R⁷ is not equal to H, where this is preferably a straight-chain alkyl or alkoxy group having 1 to 10 C atoms or a branched or cyclic alkyl or alkoxy group having 3 to 10 C atoms.

In a further preferred embodiment, R³ in the electroluminescent device according to the invention is equal to H, F, a group W which is linked to Ar², or, together with R², forms a divalent unit —CH₂—(C(R⁷)₂)ₕ—CH₂—, where h is equal to 1, 2 or 3, preferably 2. It is preferred here that only one of the radicals R⁷ is not equal to H, where this is preferably a straight-chain alkyl or alkoxy group having 1 to 10 C atoms or a branched or cyclic alkyl or alkoxy group having 3 to 10 C atoms.

In a further preferred embodiment, R⁴ in the electroluminescent device according to the invention is equal to Ar².

In a further preferred embodiment, the compound of the formula (1) or (2) in the electroluminescent device according to the invention is a compound from the following formulae:

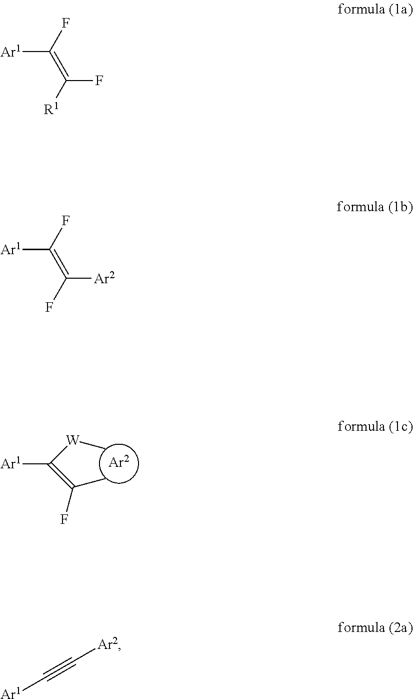

where the symbols used have the same meanings as described above.

In a further preferred embodiment of the electroluminescent device according to the invention, Ar¹ and/or Ar² is a phenyl group, which may be substituted by a radical R⁶.

In a further preferred embodiment of the electroluminescent device according to the invention, R⁶ is equal to F, a straight-chain alkyl or alkoxy group having 1 to 20 C atoms or a branched or cyclic alkyl or alkoxy group having 3 to 20 C atoms, or an aromatic ring system having 6 to 10 aromatic ring atoms, each of which may be substituted by one or more radicals R⁸.

In a further preferred embodiment of the electroluminescent device according to the invention, R⁸ is equal to F or a straight-chain alkyl or alkoxy group having 1 to 10 C atoms or a branched or cyclic alkyl or alkoxy group having 3 to 10 C atoms.

In a further preferred embodiment of the electroluminescent device according to the invention, W is a group —O—, —S—, —(NR′)— or —(C(R⁷)₂)—, in which R⁷ is preferably H, where the group W is particularly preferably —(CH₂)₂—.

The compound of the formula (3) is preferably one of the following compounds having the formulae (6) to (20), where the bonding to the double or triple bond of the formula (1) or (2) can take place at any desired and chemically possible site:

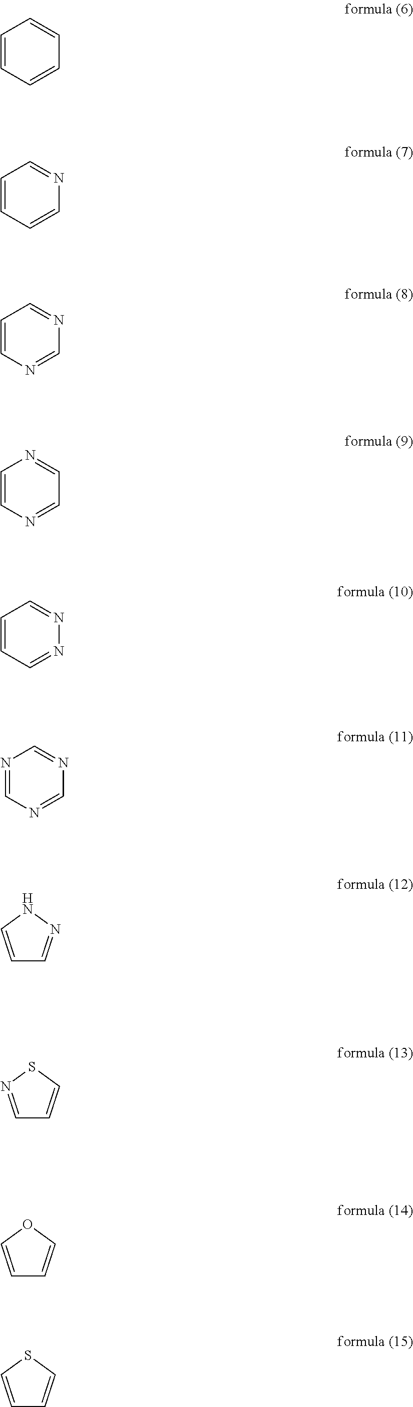

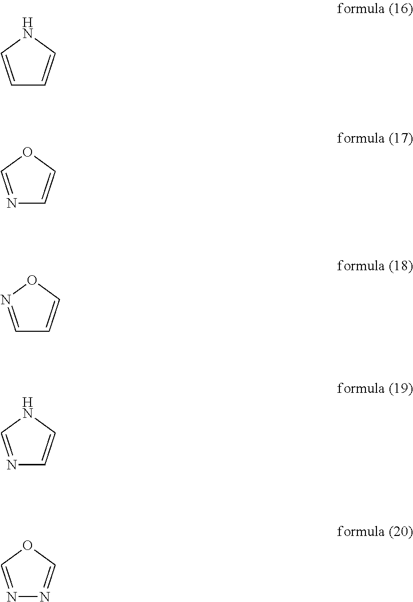 [SMILES: c1cc[nH]c1 ; c1cocn1 ; c1cnoc1 ; c1c[nH]cn1 ; c1nnco1]

The compound of the formula (4) is preferably a compound which arises from the chemically conceivable condensation of two compounds having the formulae (6) to (20), where the bonding to the double or triple bond of the formula (1) or (2) can take place at any desired and chemically possible site.

The compound of the general formula (1) or (2) is preferably one of the following compounds:

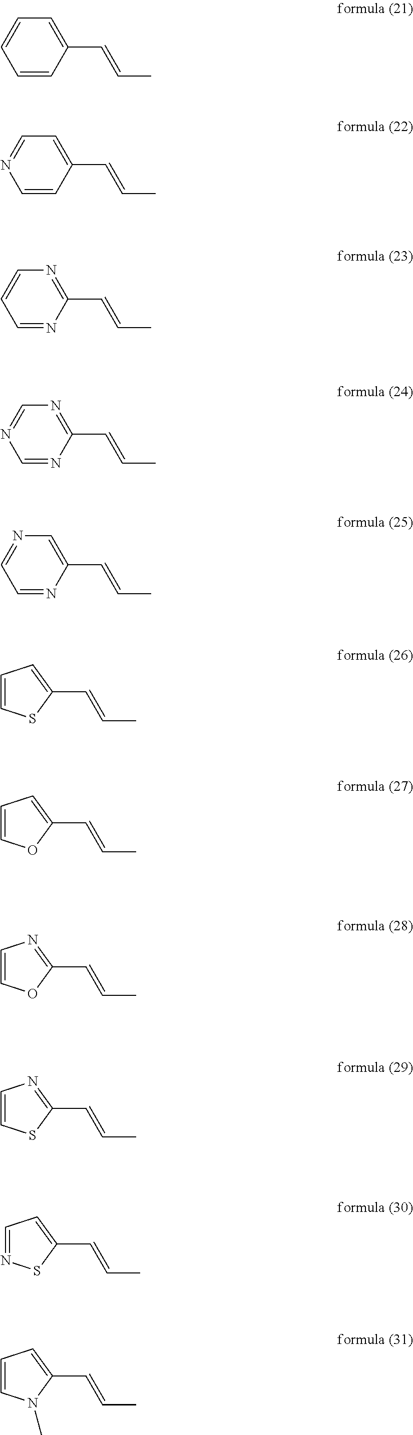

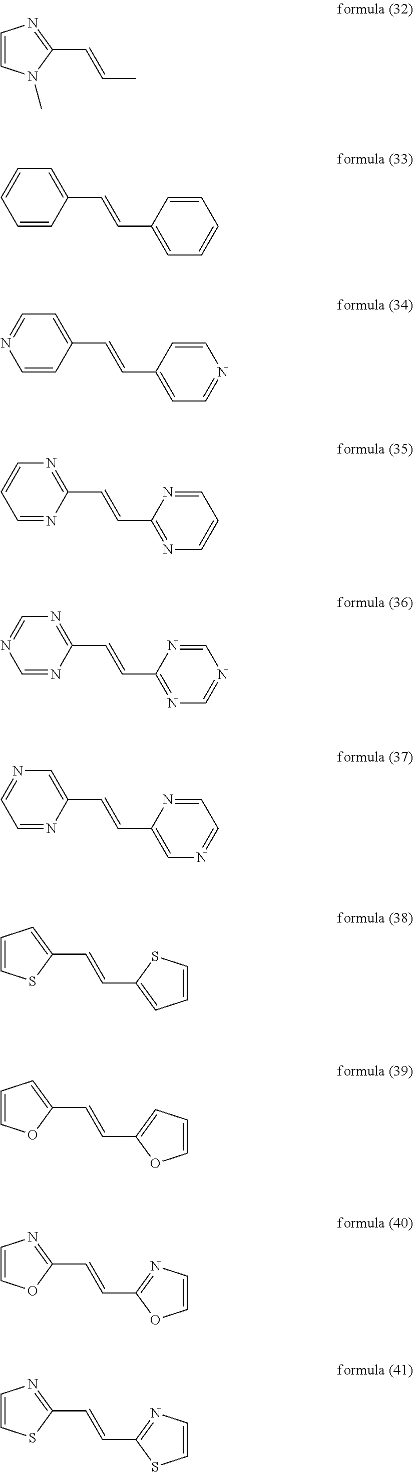

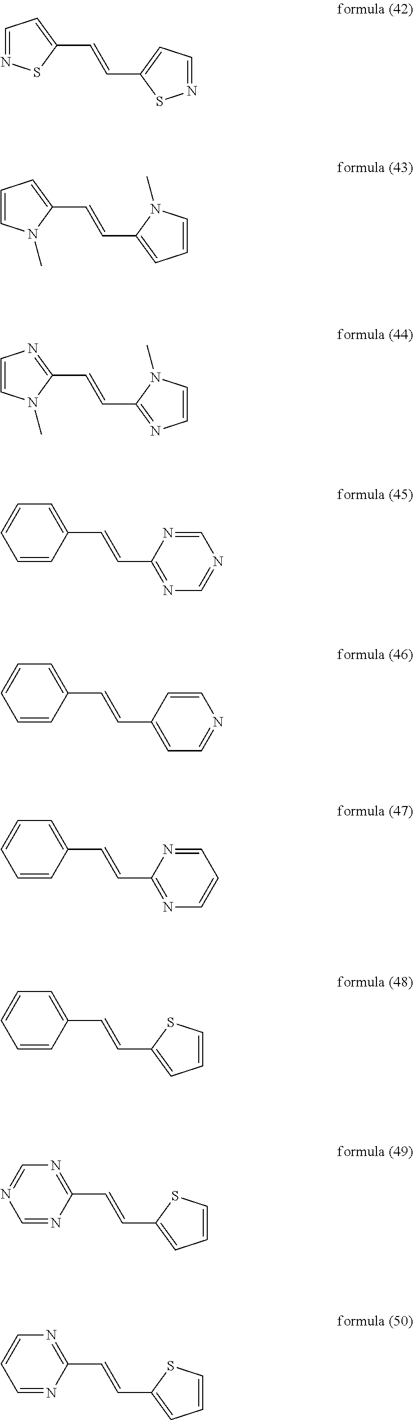

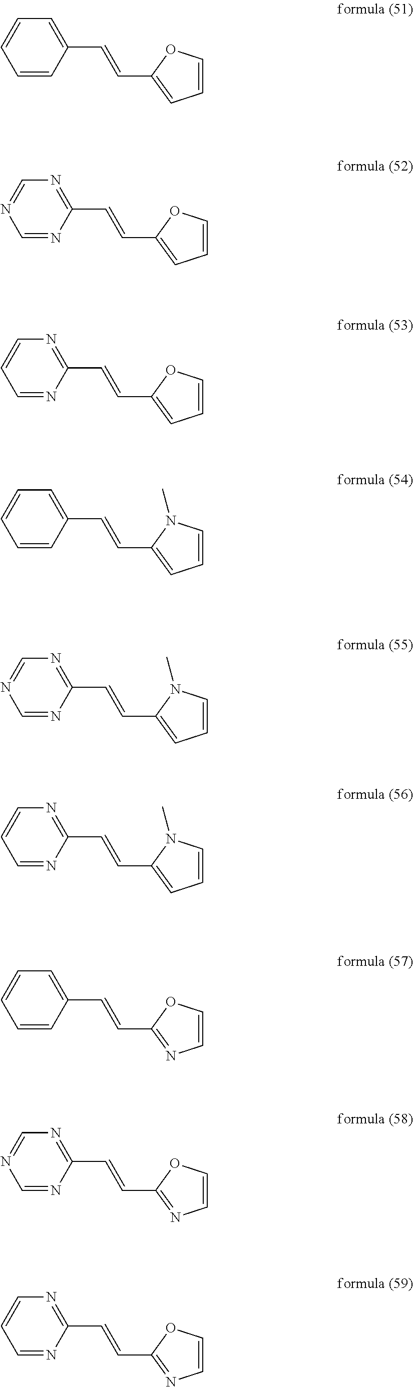

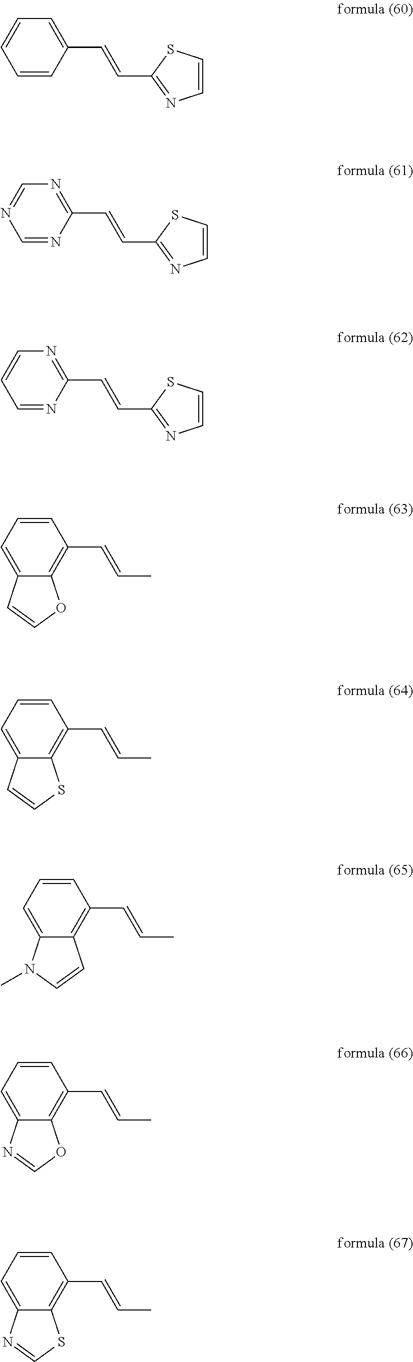

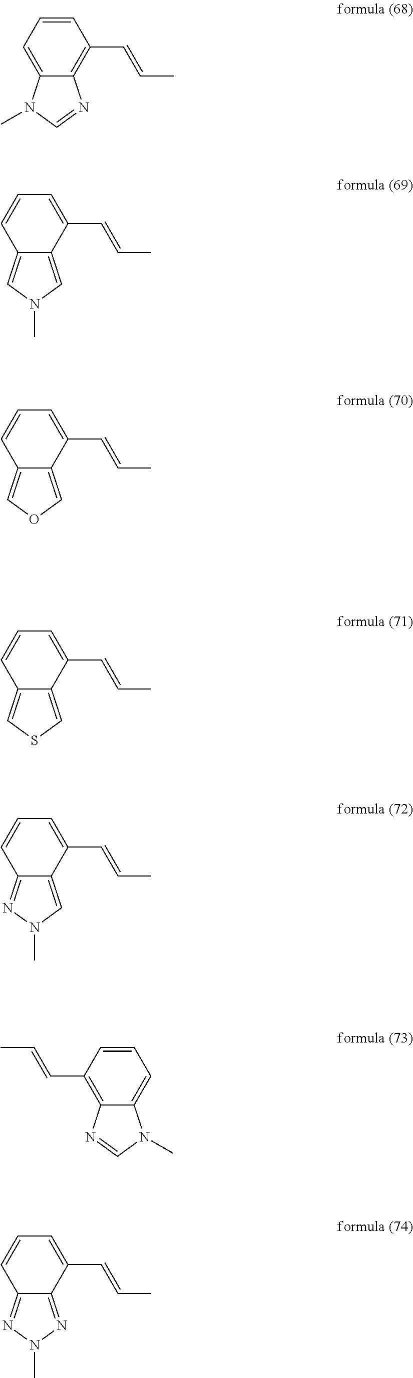

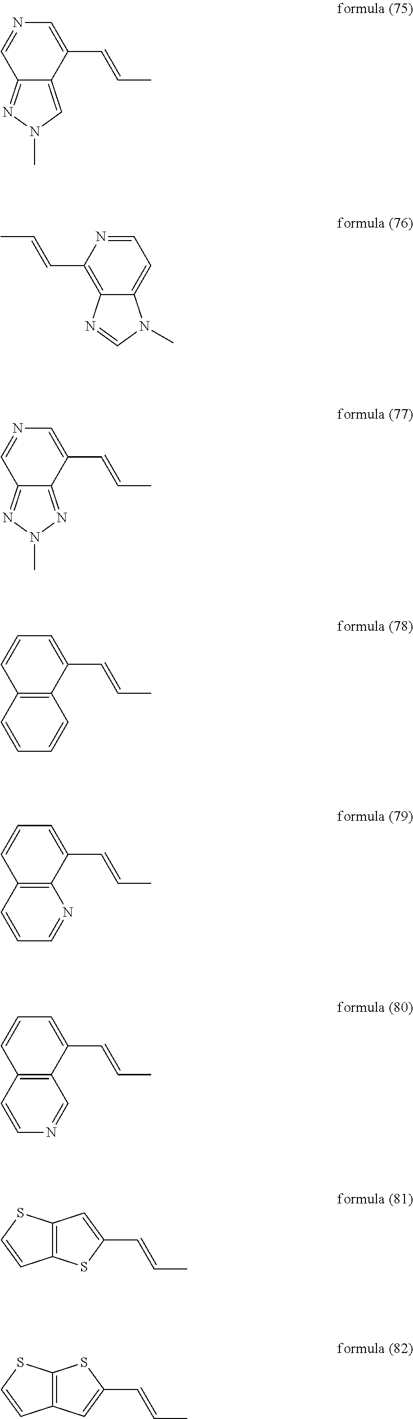

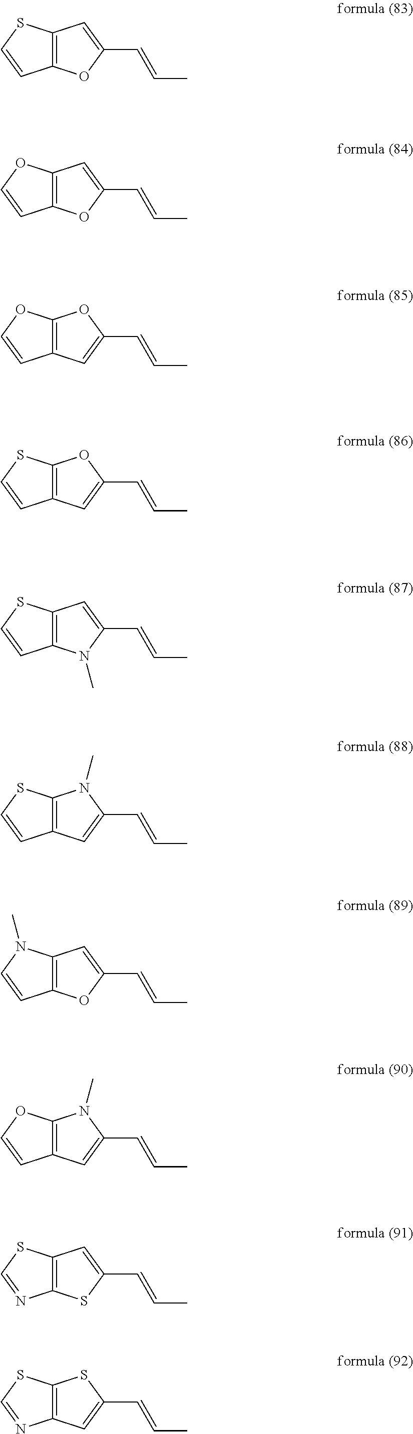

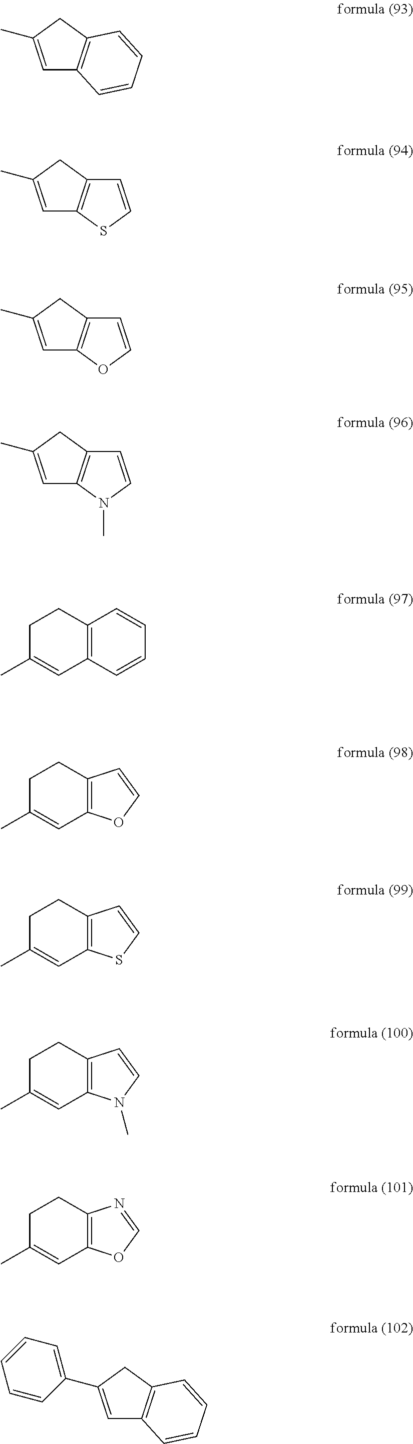

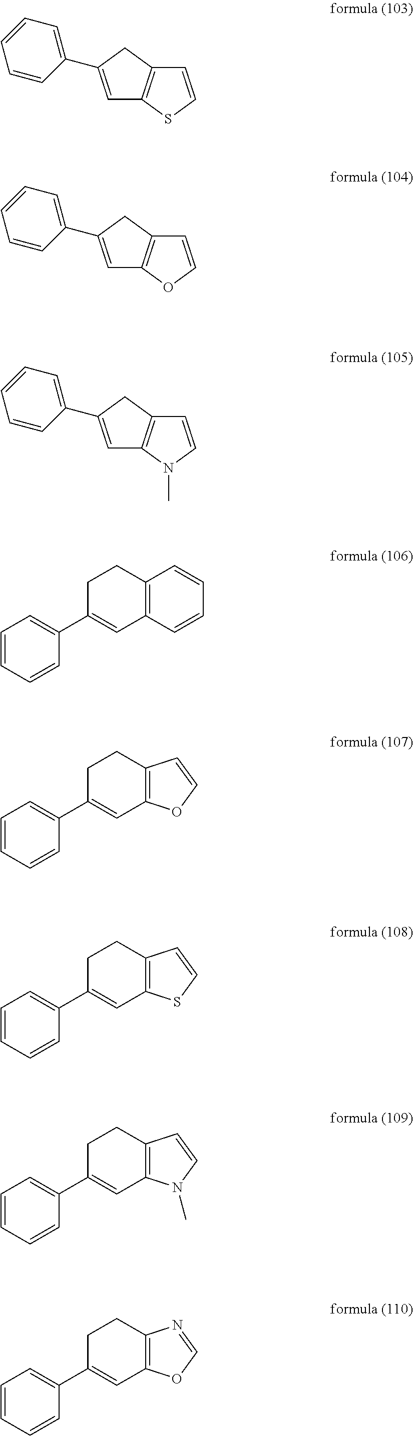

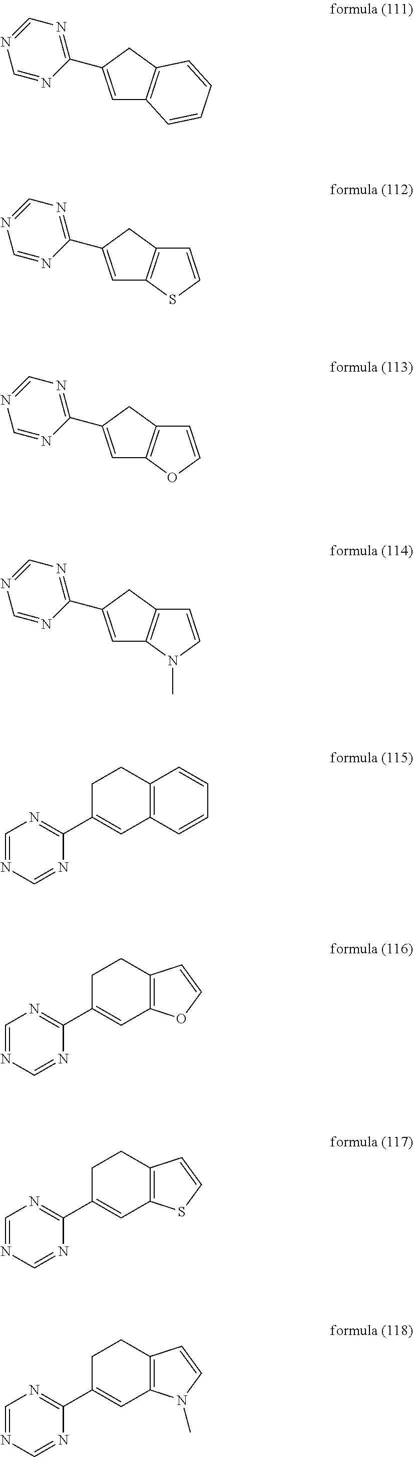

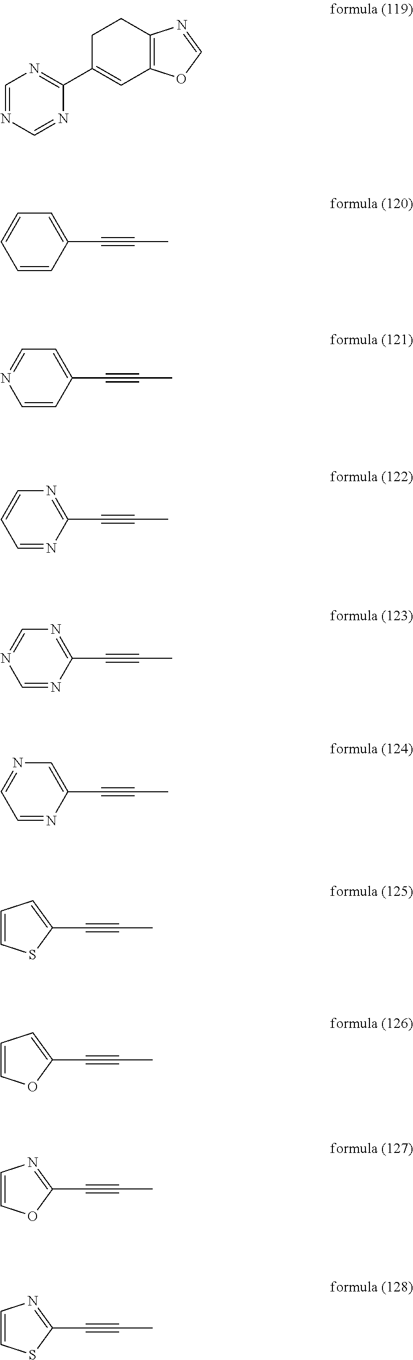

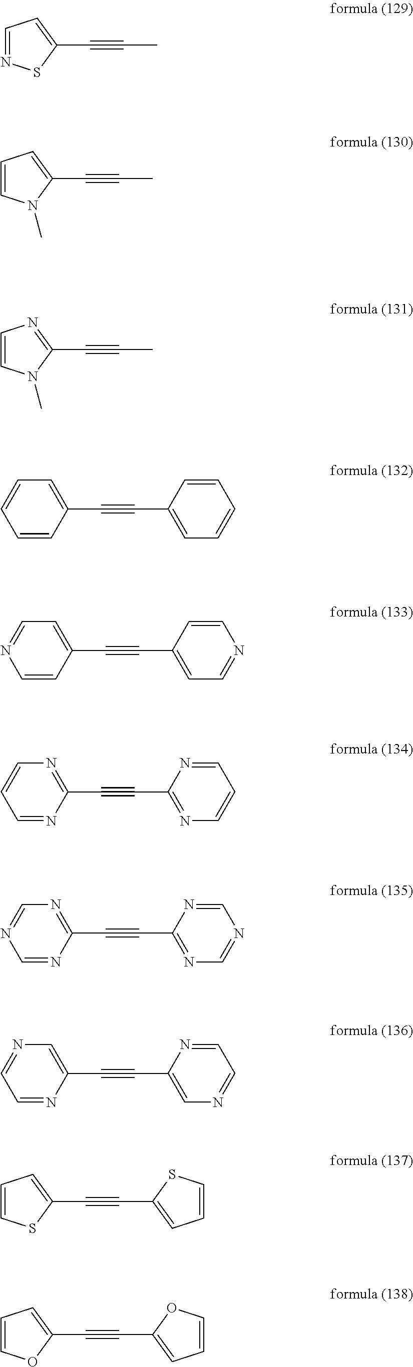

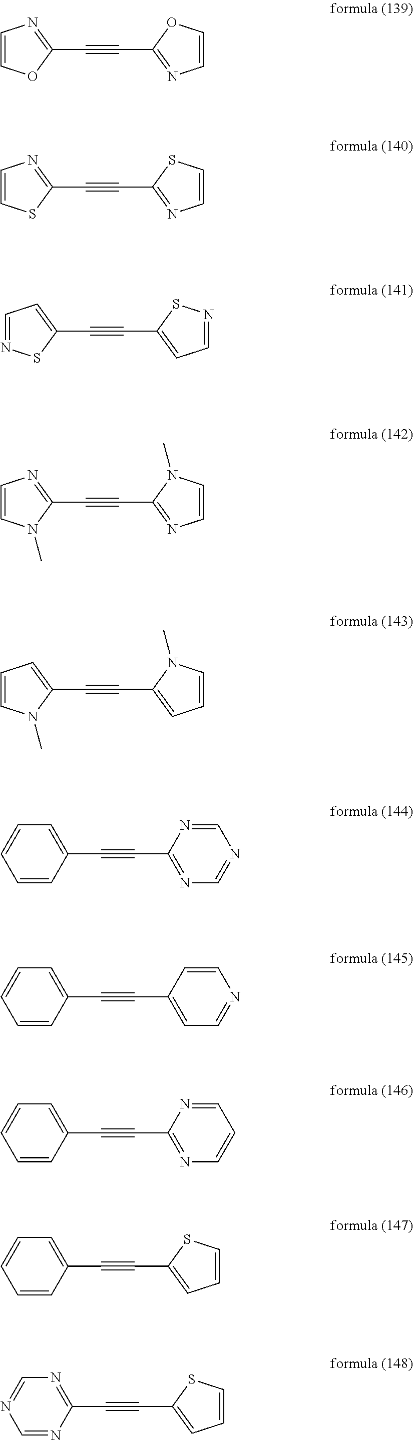

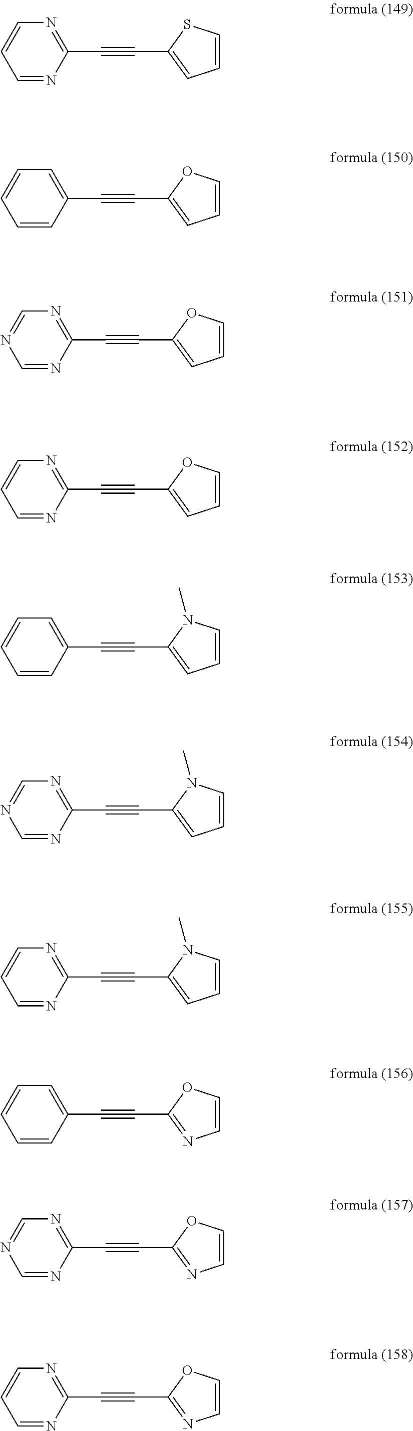

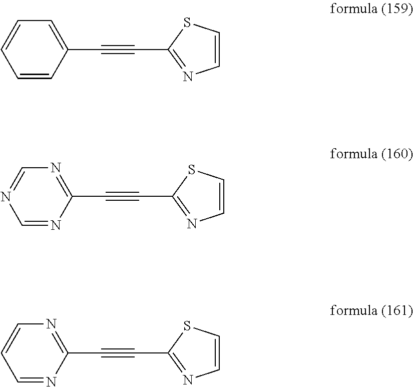

where one or more H atoms of these compounds may be replaced by a radical R¹, which is defined as above, but in this case is different from H.

The compounds of the formula (1) or (2) preferably contain one, two, three or four of the radicals R¹, but preferably one or two radicals R¹, which are different from H.

The compounds of the formula (1) or (2) are particularly preferably selected from the following compounds:

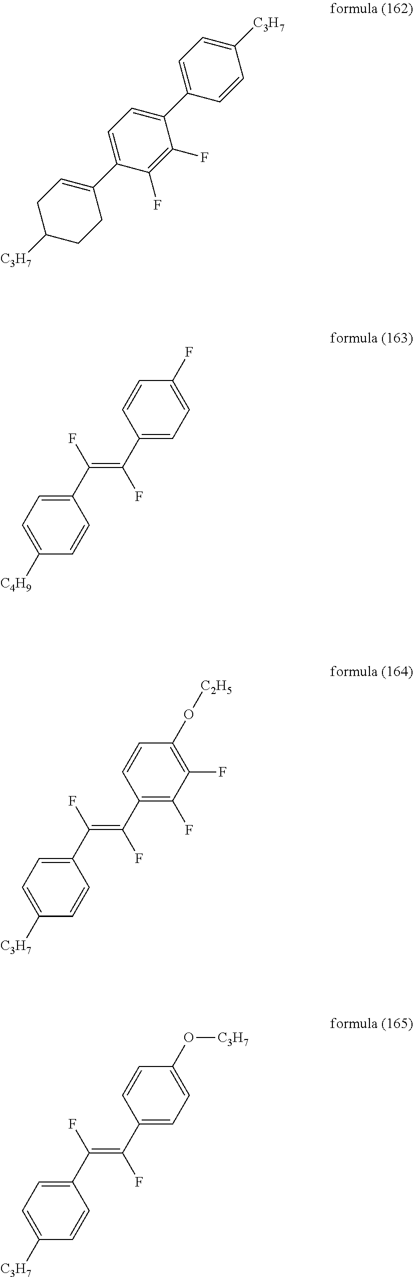

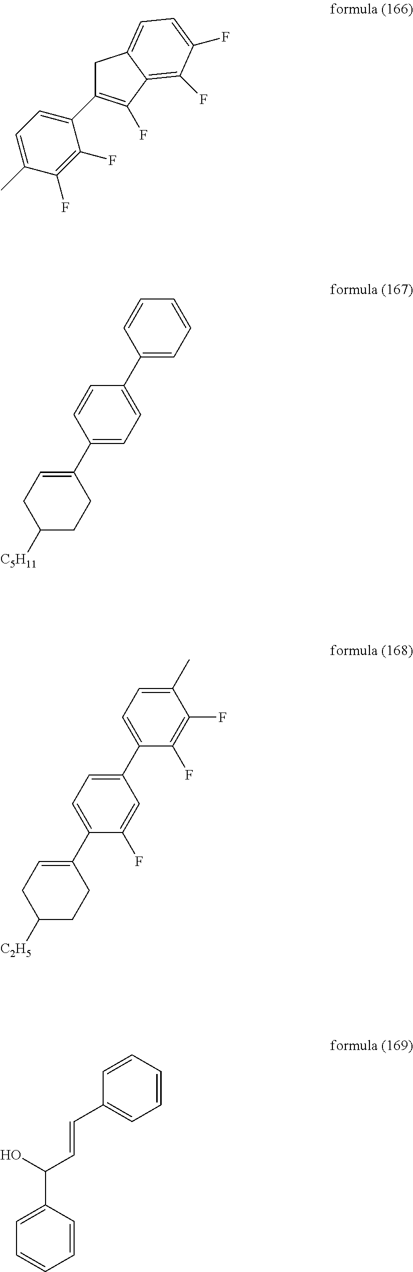

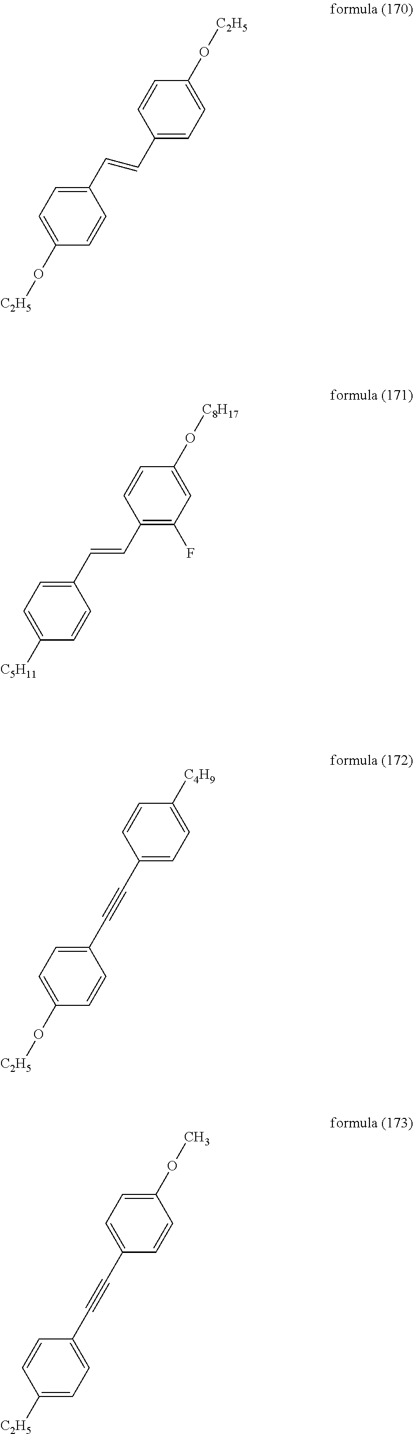

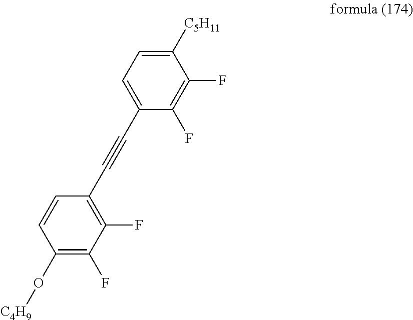

The compound of the formula (1) or (2) is preferably a fluorescent emitter compound, i.e. the compound emits radiation from the electronically excited singlet state. However, it is also conceivable for the compound to be a phosphorescent compound which emits radiation from the electronically excited triplet state. The compound of the formula (1) or (2) is preferably employed as UV light-emitting compound, i.e. the electroluminescent device according to the invention is preferably a UV light-emitting device.

In a further preferred embodiment, the compound of the formula (1) or (2) is an emitter compound which has a high oscillator strength, preferably one of greater than 0.1, more preferably greater than 0.2 and most preferably greater than 0.5.

Oscillator strength, f, here is taken to mean a quantity which characterises the strength of the coupling of a transition between two certain quantum states (for example P. W. Atkins and R. S. Friedman, Molecular Quantum Mechanics, Oxford Univ. Press, third ed.). In this, it characterises the strength of the coupling between the ground state and the excited state of a molecule. The oscillator strength gives an indication of the efficiency of optical or electrical excitations. However, different determination methods also give different values for the oscillator strength. A meaningful comparison of oscillator strengths is only possible if the values have been determined using the same method. The method used herein is described in the examples.

In an embodiment, the emitting layer of the device according to the invention comprises an emitter of the formula (1) or (2) and at least one host material. The host material here has a larger band gap (=separation between valence band (LUMO—lowest unoccupied molecular orbital) and conduction band (HOMO—highest occupied molecular orbital)) or a higher excited electronic state. The host material consequently has a higher S₁ or T₁ level, preferably the S₁ level of the host material is higher than that of the emitter. S₁ here is the first electronically excited singlet level. T₁ is the first electronically excited triplet level.

The above-mentioned materials can be employed as emitters in the emission layer. However, the materials of the formula (1) or (2) can also be employed as host materials. The host compound of the formula (1) or (2) can be doped either with at least one dopant (emitter) of any desired type or with at least one emitter of the formula (1) or (2). The present invention therefore also relates to an electroluminescent device which is characterised in that the emission layer comprises at least one compound of the formula (1) or (2) as host material in the emission layer.

The present invention therefore also relates to an electroluminescent device which is characterised in that the emission layer comprises at least one compound of the formula (1) or (2) as host material and at least one compound of the formula (1) or (2) as emitter in the emission layer.

In a further embodiment of the present invention, particular preference is given to the use of polystyrene or of derivatives of polystyrene as host material.

In a further embodiment, the emitting layer comprises at least one further UV emitter and/or at least one further host material.

Suitable materials for the emitting layer, either as emitter or as host, are compiled by way of example in the following table with the corresponding references.

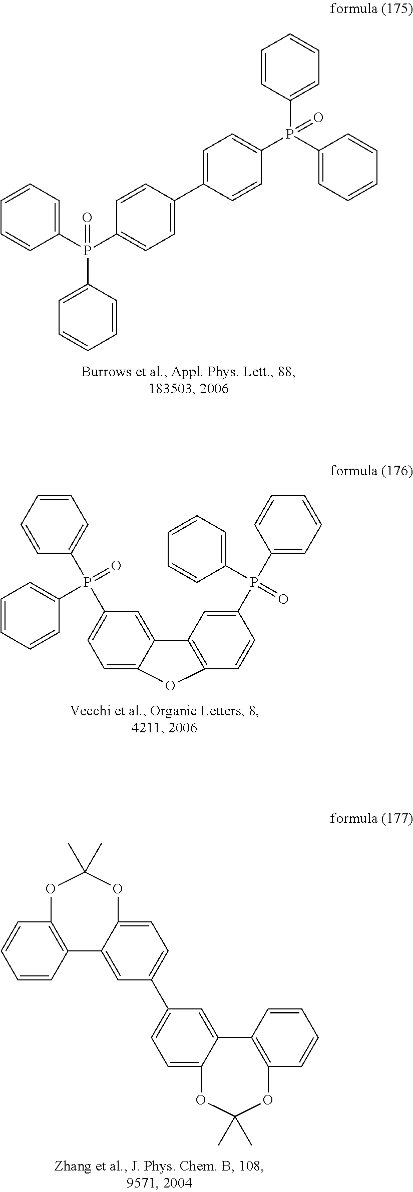 [SMILES: O=P(c1ccccc1)(c1ccccc1)c1ccc(-c2ccc(P(=O)(c3ccccc3)c3ccccc3)cc2)cc1 ; O=P(c1ccccc1)(c1ccccc1)c1ccc2oc3ccc(P(=O)(c4ccccc4)c4ccccc4)cc3c2c1 ; CC1(C)Oc2ccccc2-c2cc(-c3ccc4c(c3)-c3ccccc3OC(C)(C)O4)ccc2O1]

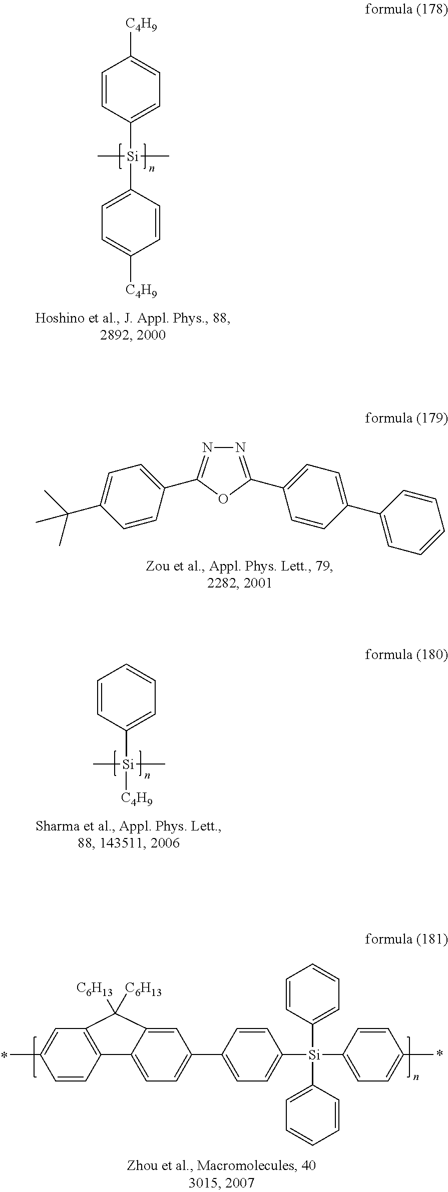

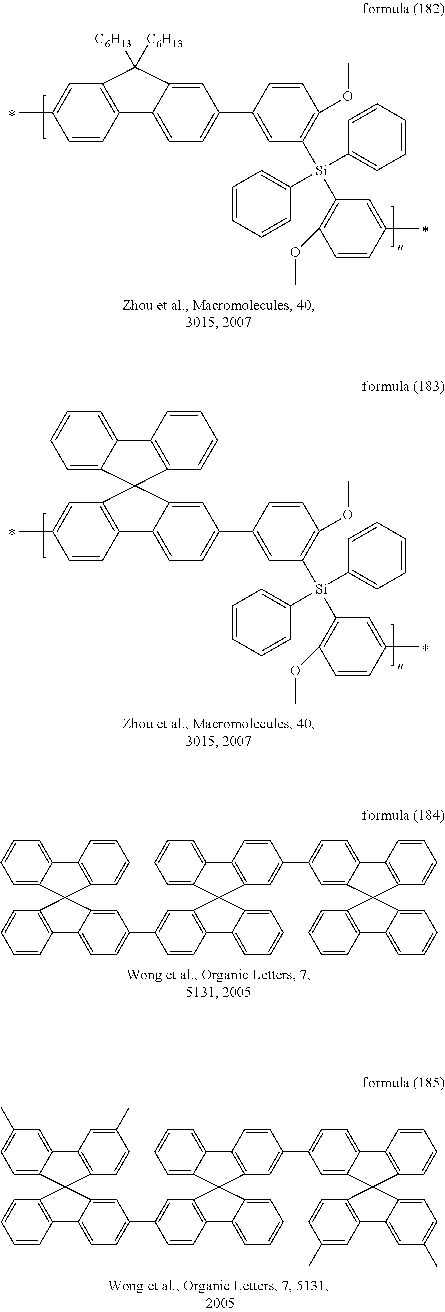

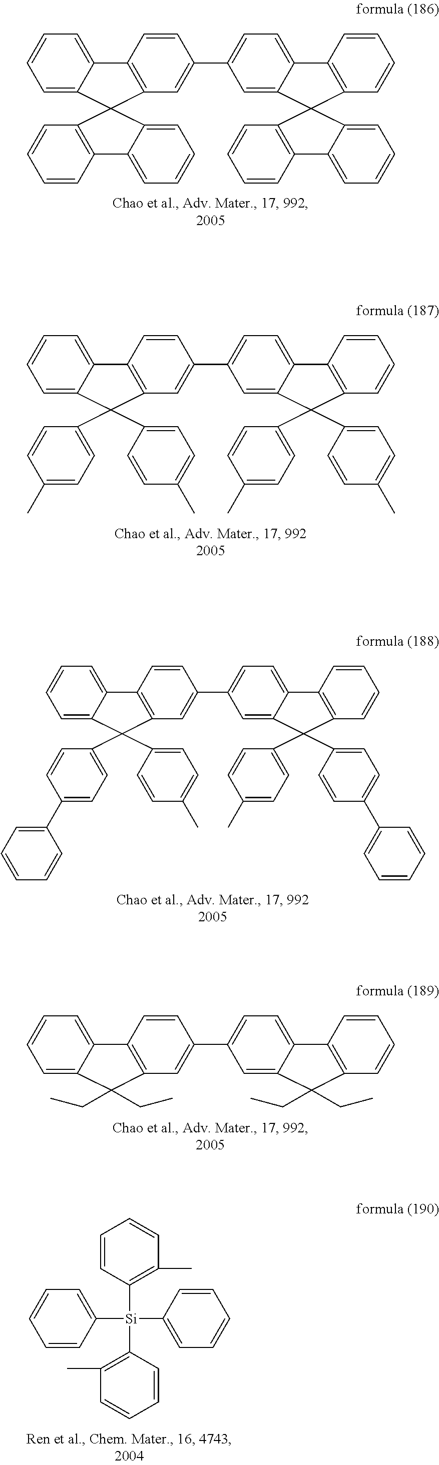

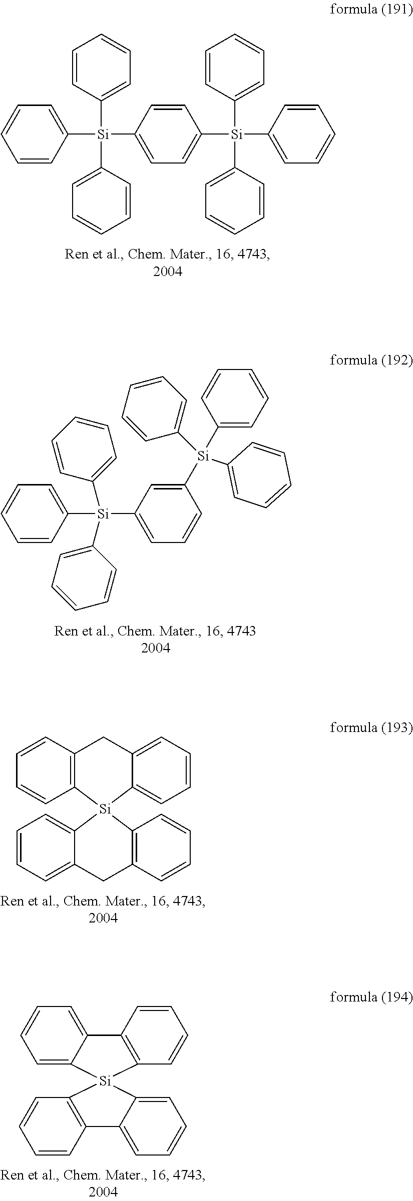 [SMILES: c1ccc([Si](c2ccccc2)(c2ccccc2)c2ccc([Si](c3ccccc3)(c3ccccc3)c3ccccc3)cc2)cc1 ; c1ccc([Si](c2ccccc2)(c2ccccc2)c2cccc([Si](c3ccccc3)(c3ccccc3)c3ccccc3)c2)cc1 ; c1ccc2c(c1)Cc1ccccc1[Si]21c2ccccc2Cc2ccccc21 ; c1ccc2c(c1)-c1ccccc1[Si]21c2ccccc2-c2ccccc21]

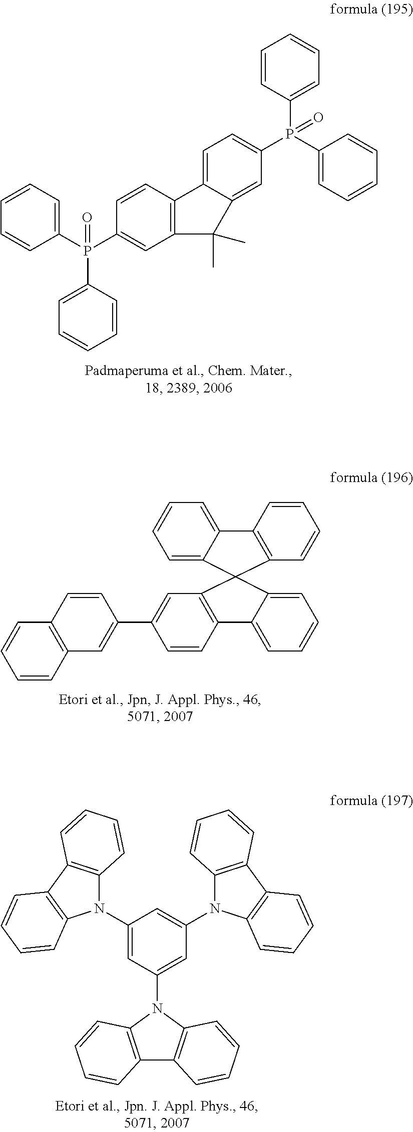

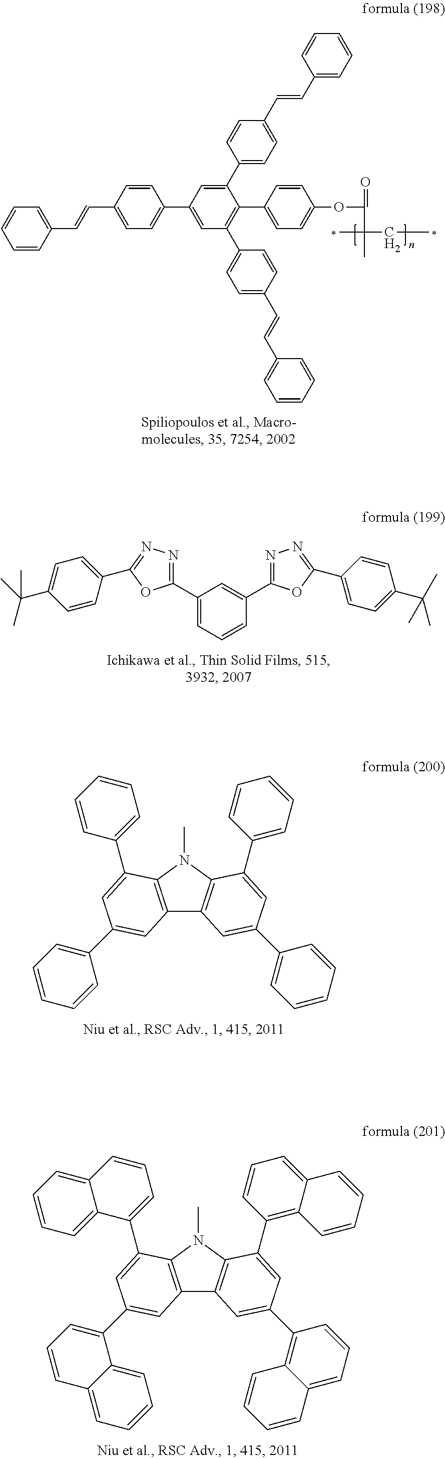

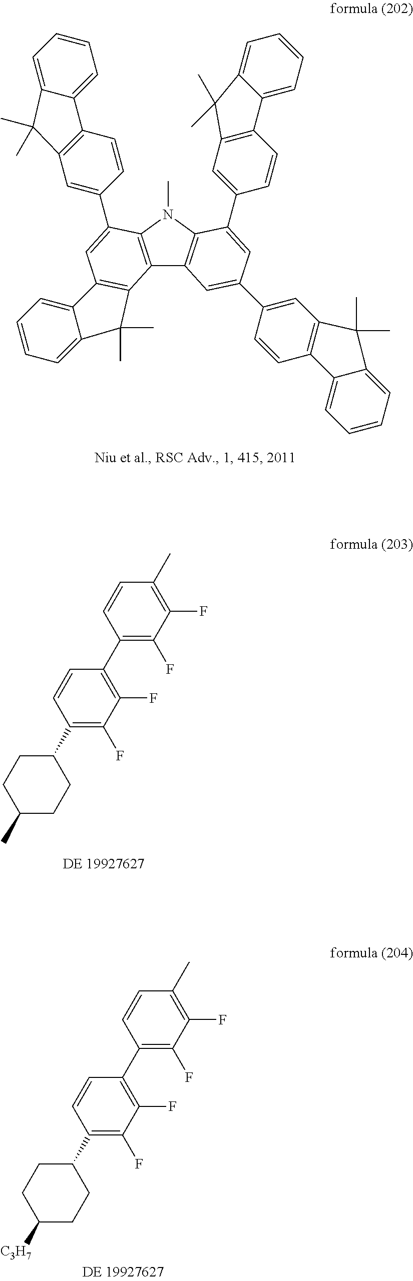

The amount of the compound of the formula (1) or (2) in the emitting layer is preferably in the range from 1 to 60% by weight, more preferably 5 to 50% by weight and even more preferably 15 to 45% by weight, based on the total weight of all constituents of the emitting layer.

The amount of the host material in the emitting layer is preferably in the range from 40 to 99% by weight, more preferably 50 to 95% by weight and even more preferably 55 to 85% by weight, based on the total weight of all constituents of the emitting layer.

The performance data of the devices according to the invention can be improved further in various ways.

As already mentioned, at least one host material is usually used in the emission layer of organic electroluminescent devices besides the emitter or emitters. However, particularly good results can be achieved on use of a so-called mixed host in the device according to the invention.

In a further preferred embodiment, a mixed host is used in the emission layer of the device according to the invention. This enables the radiation intensities of the devices to be significantly increased and the operating voltages to be significantly reduced. Mixed host means that the host consists of at least 2 different compounds. The person skilled in the art will be able to fall back here without difficulties on a multiplicity of host compounds known in the prior art.

In a further embodiment, further layers are introduced between the emitting layer and one of the two electrodes.

It is advantageous if at least one blocking layer is used between emitting layer and one of the electrodes. This enables, in particular, the operating voltage to be reduced and the absolute radiation intensities to be increased. Suitable blocking layers can block excitons, electrons or holes.

The present invention therefore also relates to a device, as disclosed herein, which preferably comprises a further layer between the emitting layer and one of the two electrodes, characterised in that the additional layer comprises an exciton-blocking material (blocking material) having a band gap of 3.4 eV or higher, preferably 3.6 eV or higher, very preferably 3.8 eV or higher and very particularly preferably 4.0 eV or higher.

The present invention also relates to a device, as disclosed herein, which comprises a further layer between the emitting layer and one of the two electrodes, characterised in that the additional layer comprises a hole-blocking material (barrier material) having an HOMO of lower than −5.9 eV, preferably lower than −6.0 eV, very preferably lower than −6.2 eV and very particularly preferably lower than −6.3 eV.

The present invention also relates to a device, as disclosed herein, which preferably comprises a further layer between the emitting layer and one of the two electrodes, characterised in that the additional layer comprises an electron-blocking material (blocking material) having an LUMO of higher than −2.2 eV, preferably higher than −2.1 eV.

In a very preferred embodiment, the device according to the invention comprises a blocking layer which blocks both excitons and also holes.

In a further very preferred embodiment, the device according to the invention comprises a blocking layer which blocks both excitons and also electrons.

In a very preferred embodiment, the blocking layer is formed by crosslinking one or more compounds containing at least 2 or more crosslinkable groups (hereafter precursor). Especial preference is given to a blocking layer which is formed by precursors of the compound of the formula (1) or (2), which furthermore contain at least 2 or more crosslinkable groups.

A crosslinkable group is a group containing a crosslinkable reagent which results in a crosslinking reaction with the aid of heat, radiation or both. The radiation source can be an electron beam or UV radiation. The preferred UV radiation source emits radiation of a wavelength of 200 to 400 nm, very preferably a radiation of 300 to 400 nm. Suitable sources for UV radiation are, for example, mercury UV fluorescent lamps, UV LEDs and UV laser diodes.

Suitable crosslinkable groups are, for example, the acrylate group (for example Scheler et al., In Macromol. Symp. 254, 203-209 (2007)), the vinyl or styrene group (for example WO 2006/043087) and the oxetane group (for example Mueller et al., In Nature 421, 829-833 (2003)).

In a preferred embodiment, the precursor compound for the blocking layer is a compound of the general formula (205).

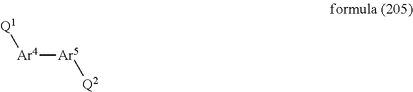

where Ar⁴ and Ar⁵ each, independently of one another, denote an aromatic or heteroaromatic 5- or membered ring and preferably represent a ring of the formula (3) defined above, and Q¹ and Q² is each, independently of one another, a crosslinkable group, which is preferably selected from the following formula (206) to (229):

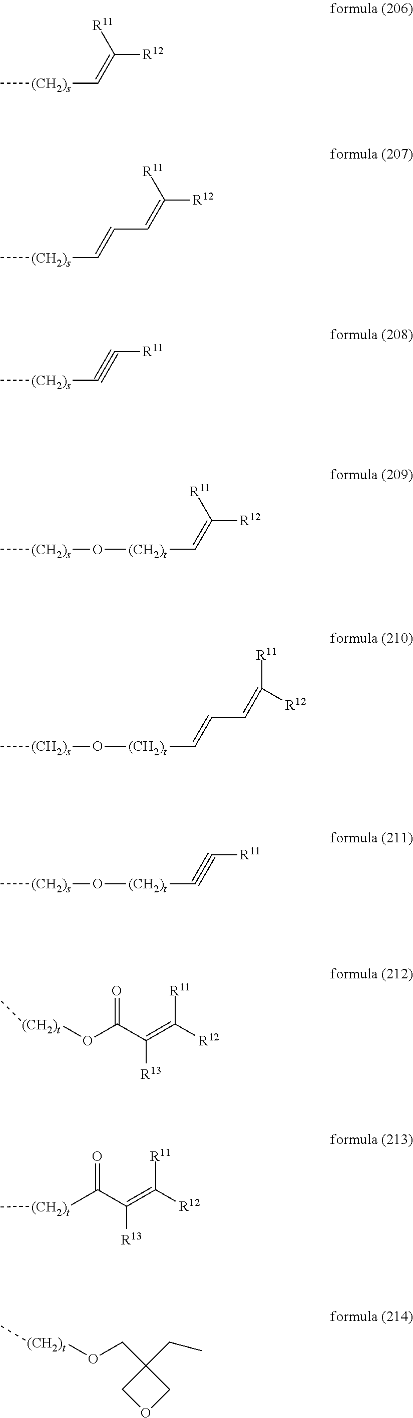

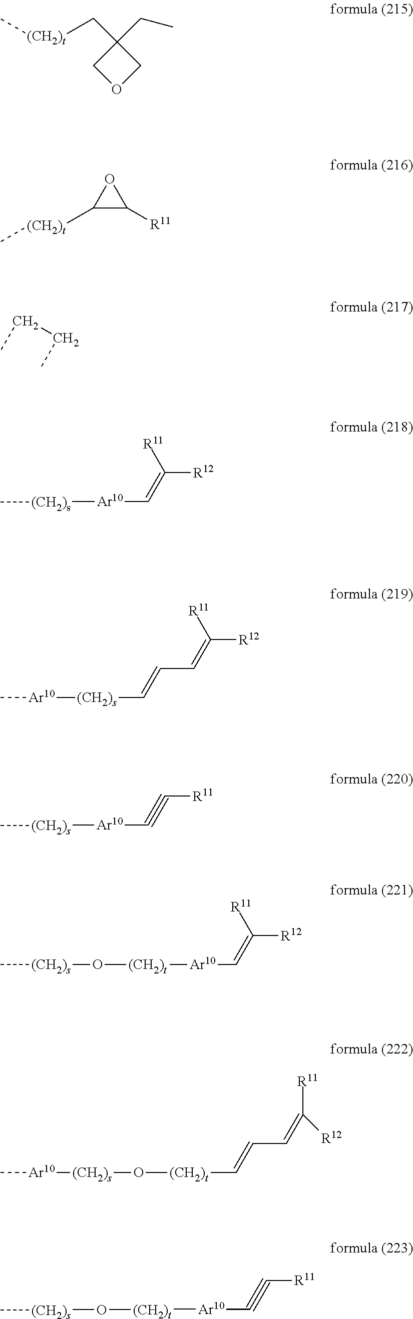

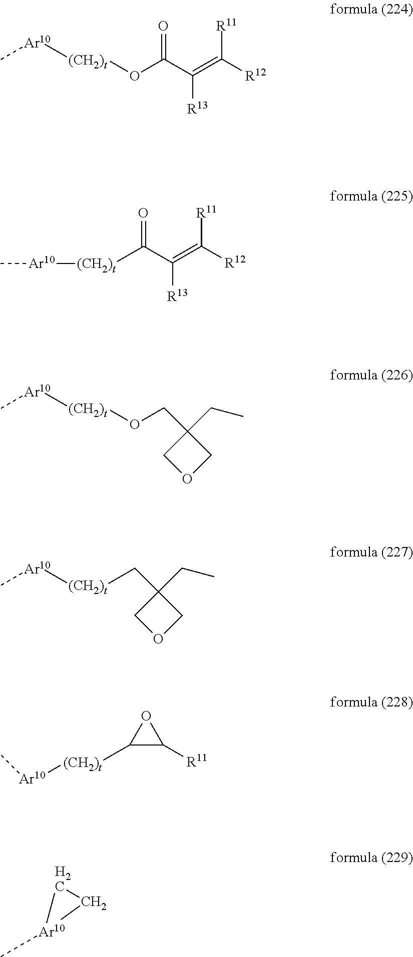

where
the radicals R¹¹, R¹² and R¹³ are on each occurrence, identically or differently, H, a straight-chain or branched alkyl group having 1 to 6 C atoms;
Ar¹⁰ in the formulae (218) to (229) is a mono- or polycyclic, aromatic or heteroaromatic ring system having 5 or 6 ring atoms, which may be substituted by one or more radicals R, where R is on each occurrence, identically or differently, H, D, F, an aliphatic hydrocarbon radical having 1 to 20 C atoms, an aromatic hydrocarbon radical having 6 to 20 aromatic ring atoms or a heteroaromatic hydrocarbon radical having 5 to 20 aromatic ring atoms, in which, in addition, one or more H atoms may be replaced by F;
where two or more substituents R may also form a mono- or polycyclic, aliphatic or aromatic ring system with one another;
s is an integer from 0 to 8;
t is an integer from 1 to 8;
and where the dashed bond represent the linking of the crosslinkable group to one of the mono- or polycyclic, aromatic or heteroaromatic ring systems Ar⁴ or Ar⁵ in formula (205).

In the group of the formula (217), the two dashed lines mean that Ar⁴ and/or Ar⁵ in the compound of the formula (205) are connected in the ortho position to the two carbon atoms of the ethylene group, so that a four-membered ring forms. Analogously, Ar¹⁰ in the group of the formula (229) is connected to the two carbon atoms of the ethylene group in the ortho position, so that a four-membered ring forms.

Examples of preferred precursor compounds for the blocking layer in accordance with the embodiments indicated above are the compounds of the following structures.

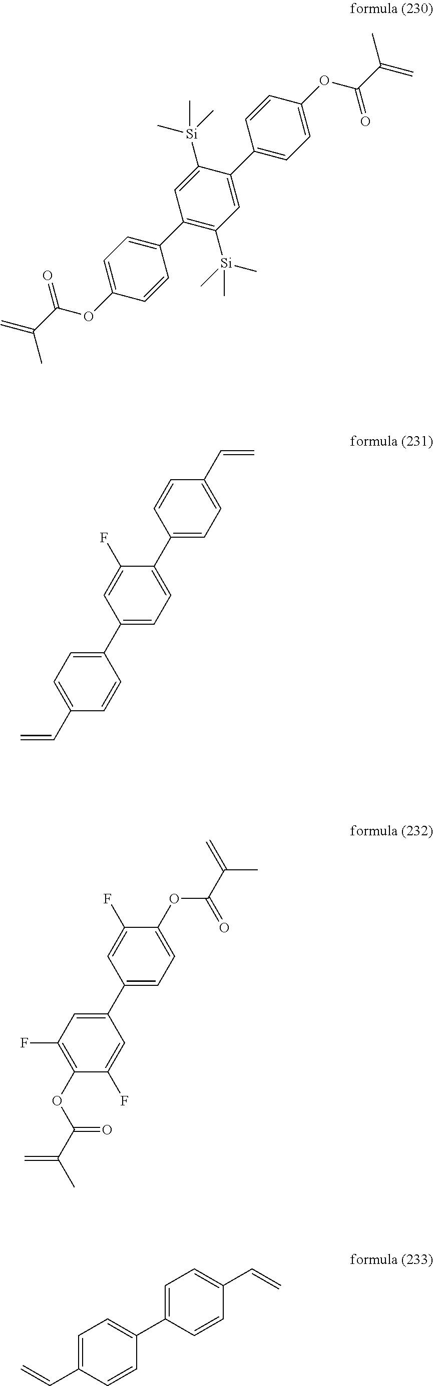

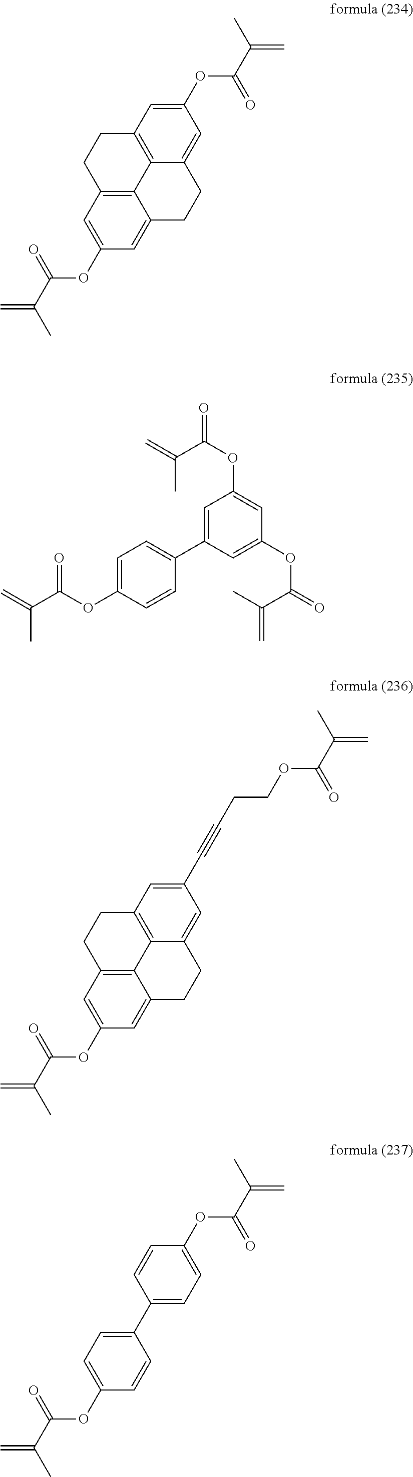

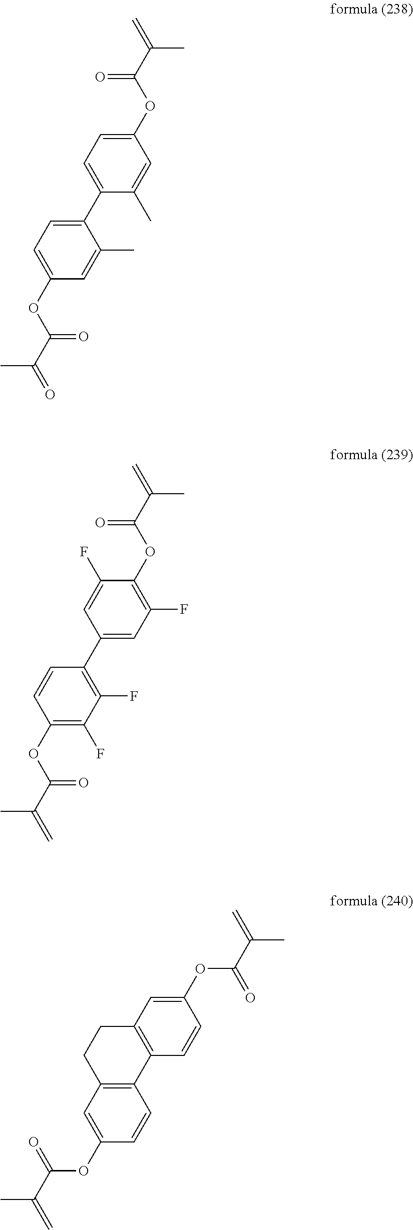

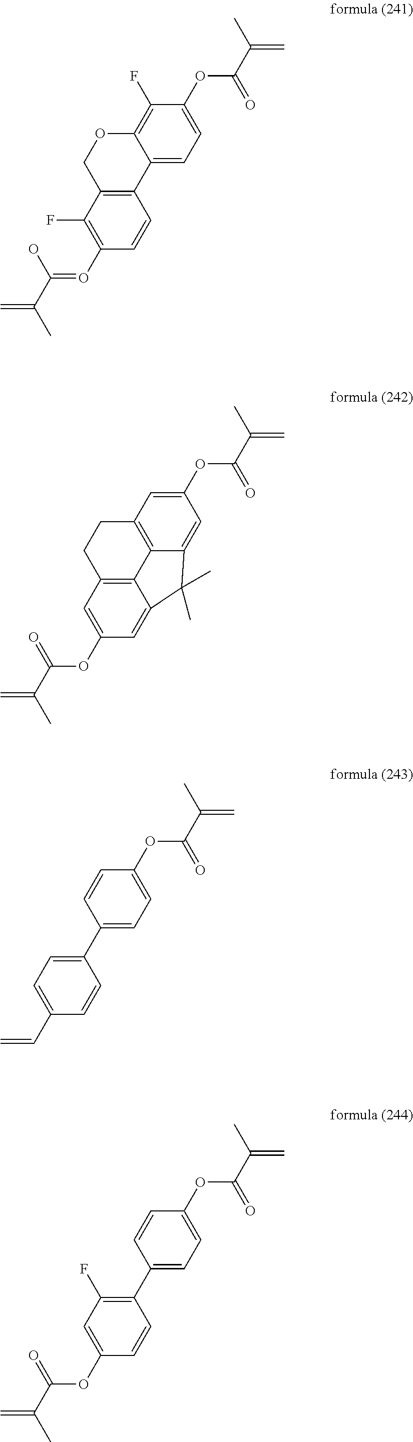

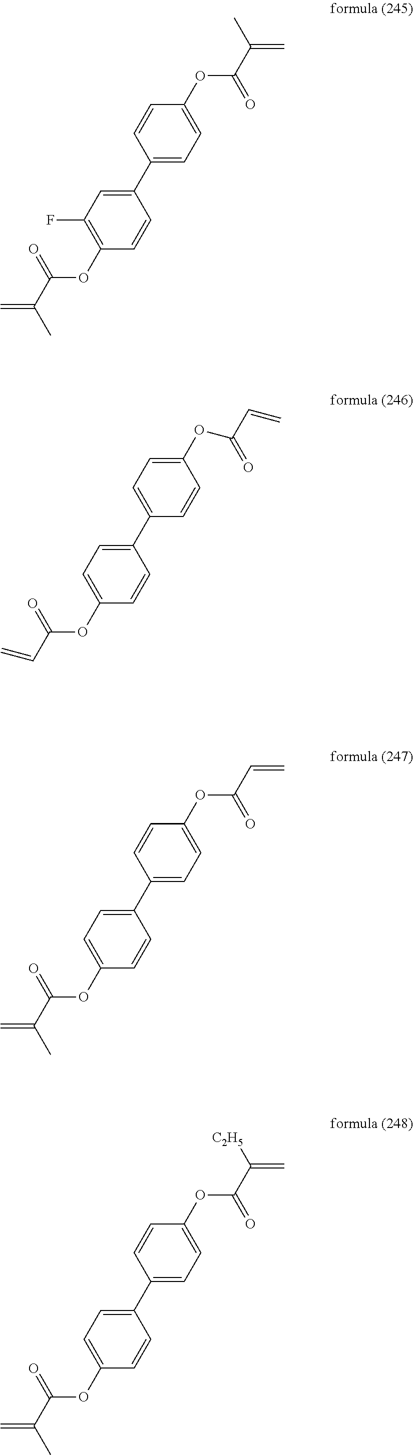

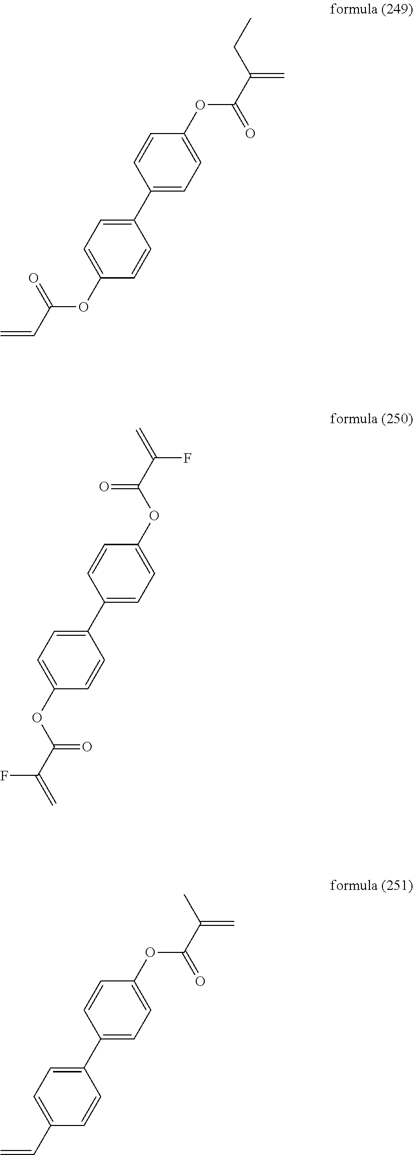

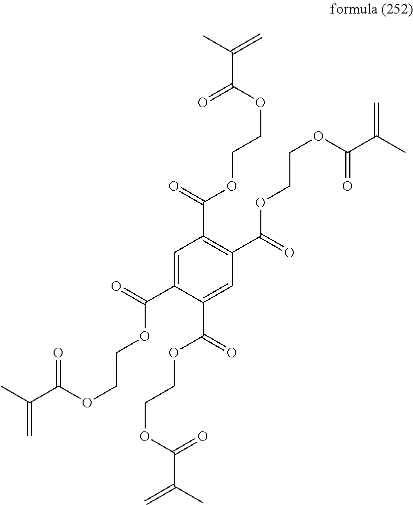 [SMILES: C=C(C)C(=O)OCCOC(=O)c1cc(C(=O)OCCOC(=O)C(=C)C)c(C(=O)OCCOC(=O)C(=C)C)cc1C(=O)OCCOC(=O)C(=C)C]

Processes for the preparation of the said precursor compounds are well known to the person skilled in the art from the prior art (for example WO 2010/133278 and U.S. Pat. No. 7,807,068).

In a furthermore preferred embodiment, the electroluminescent device according to the invention emits radiation having a wavelength in the range from 280 nm and 380 nm.

The electroluminescent device can be any electroluminescent device. The person skilled in the art will be able to make a selection here without difficulties from a large number of devices known to him. The electroluminescent device is preferably an organic light-emitting diode (OLED), polymeric light-emitting diode (PLED), organic light-emitting electrochemical cell (OLEC, LEC or LEEC), an organic light-emitting transistor (O-LETs) and an organic light-emitting electrochemical transistor. In a very preferred embodiment, the present invention relates to OLEDs or PLEDs. In a furthermore very preferred embodiment, the present invention relates to OLECs.

The electroluminescent device comprises cathode, anode and at least one emitting layer. Apart from these layers, it may also comprise further layers, for example in each case one or more hole-injection layers, hole-transport layers, hole-blocking layers, electron-transport layers, electron-injection layers, exciton-blocking layers and/or charge-generation layers. Interlayers, which have, for example, an exciton-blocking function, may likewise be introduced between two emitting layers. However, it should be pointed out that each of these layers does not necessarily have to be present. The electroluminescent device may comprise one emitting layer, or it may comprise a plurality of emitting layers, where it is preferred if it comprises one emitting layer.

In a preferred embodiment, the electroluminescent device according to the invention comprises a hole-injection layer, which is also called buffer layer. The work function of the hole-injection layer is greater than 5.0 eV, preferably greater than 5.4 eV, very preferably greater than 5.8 eV and very particularly preferably greater than 6.0 eV. In a further embodiment, the hole-injection layer comprises conductive, conjugated polymers, such as, for example, polythiophene, polyaniline and polypyrrole and derivatives thereof. Such polymers are in some cases also commercially available, such as, for example, CLEVIOS™ P VP AI 4083, CLEVIOS™ HIL 1.3, and CLEVIOS™ HIL 1.3N from Heraeus Precious Metals GmbH & Co. KG.

The compounds of the formula (1) or (2) may also be incorporated into the side chain of polymers. The incorporation of the compounds into the side chain of polymers has various advantages, which are shown below.
1) The polymers have improved solubility in organic solvents and thus also improved processability.
2) The polymers have improved layer-formation properties.
3) The polymers have higher glass transition temperatures (Tg) compared with small molecules.
4) The polymers have a broader process window and improved performance data.

The present invention also relates to compositions comprising at least one of the compounds of the formula (1) or (2) and at least one organically functional material or an organic semiconductor selected from the group of the emitters, host materials, matrix materials, electron-transport materials (ETM), electron-injection materials (EIM), hole-transport materials (HTM), hole-injection materials (HIM), electron-blocking materials (EBM), hole-blocking materials (HBM), exciton-blocking materials (ExBM). The emitters here can be both fluorescent and phosphorescent emitters. The person skilled in the art will be able to make a selection here without difficulties from a multiplicity of known organic functional materials having the said functions. The definitions and examples of various organic functional materials can be obtained, for example, from the disclosure content of WO 2011/015265.

For the purposes of the present invention, the composition according to the invention preferably comprises at least one host material as organically functional material besides at least one compound of the formula (1) or (2) or as emitter. The composition very preferably comprises two host materials besides the at least one compound of the formula (1) or (2) as emitter. The composition very particularly preferably comprises precisely one compound of the formula (1) or (2) as emitter and two host materials. The composition furthermore very particularly preferably comprises precisely one compound of the formula (1) or (2) as emitter and precisely one host material.

The concentration of emitter(s) in the composition is preferably 1 to 60% by weight, preferably 5 to 50% by weight and most preferably 15 to 45% by weight. The total concentration of the host or host or host materials is preferably 40 to 99% by weight, preferably 50 to 95% by weight and most preferably 55 to 85% by weight.

The devices according to the invention can be produced by various processes. One or more of the layers of the electroluminescent device can be applied by means of a sublimation process, in which the materials are applied by vapour deposition in vacuum sublimation units at an initial pressure less than 10⁻⁵ mbar, preferably less than 10⁻⁶ mbar. However, it is also possible for the initial pressure to be even lower, for example less than 10⁻⁷ mbar.

A preferred process for the application of one or more layers of the electroluminescent device is the OVPD process (organic vapour phase deposition) or a process with the aid of carrier-gas sublimation, in which the materials are applied at a pressure between 10⁻⁵ mbar and 1 bar. A special case of this process is the OVJP (organic vapour jet printing) process, in which the materials are applied directly through a nozzle and thus structured (for example M. S. Arnold et al., Appl. Phys. Lett. 2008, 92, 053301).

One or more of the layers of the electroluminescent device can also be applied from solution, such as, for example, by spin coating, or by means of any desired printing process, such as, for example, screen printing, flexographic printing, offset printing, LITI (light induced thermal imaging, thermal transfer printing), ink-jet printing or nozzle printing. Soluble compounds, which are obtained, for example, by suitable substitution, are necessary for this purpose. These processes are also suitable for the application of layers comprising oligomers, dendrimers and polymers.

Likewise possible are hybrid processes, in which, for example, one or more layers are applied from solution and one or more other layers are applied by vacuum vapour deposition.

The present invention therefore also relates to a process for the production of the electroluminescent devices according to the invention by means of sublimation processes and/or by means of processes from solution.

The present invention furthermore relates to a formulation comprising a composition according to the invention and one or more solvents.

Suitable and preferred solvents are, for example, toluene, anisole, xylene, methyl benzoate, dimethylanisole, trimethylbenzene, tetralin, veratrol, tetrahydrofuran, chlorobenzene or dichlorobenzene and mixtures thereof.

Electroluminescent devices which emit blue light and/or UV radiation can be employed in a versatile manner. Applications which require light or radiation having very short wavelengths and thus represent areas of application for the devices according to the invention are found, for example, in the area of life science and medicine (for example for cell imaging) or in the area of biosensors. The devices according to the invention are furthermore used in the electronics industry, solid-state lighting and for the curing of polymers and printing ink. The present invention therefore also relates to the use of the electroluminescent devices according to the invention in the said areas.

The devices according to the invention can also be employed for the light therapy (phototherapy) of humans and/or animals. The present invention therefore furthermore relates to the use of the devices according to the invention for the treatment, prophylaxis and diagnosis of diseases by means of phototherapy. The present invention still furthermore relates to the use, of the devices according to the invention for the treatment and prophylaxis of cosmetic conditions by means of phototherapy.

Phototherapy or light therapy is used in many areas of medicine and/or cosmetics. The devices according to the invention can therefore be employed for the therapy and/or prophylaxis and/or diagnosis of all diseases and/or in cosmetic applications for which the person skilled in the art considers using phototherapy. Besides irradiation, the term phototherapy also includes photo-dynamic therapy (PDT) as well as preservation, disinfection and sterilisation in general. It is not only humans or animals that can be treated by means of phototherapy or light therapy, but also any other type of living or non-living materials. These include, for example, fungi, bacteria, microbes, viruses, eukaryotes, prokaryotes, foods, drinks, water, drinking water, cutlery, medical instruments and equipment and other devices.

The term phototherapy also includes any type of combination of light therapy and other types of therapy, such as, for example, treatment with active compounds. Many light therapies have the aim of irradiating or treating exterior parts of an object, such as, for example, the skin of humans and animals, wounds, mucous membranes, the eye, hair, nails, the nail bed, gums and the tongue. In addition, the treatment or irradiation according to the invention can also be carried out inside an object in order, for example, to treat internal organs (heart, lung, etc.) or blood vessels or the breast.

The therapeutic and/or cosmetic areas of application according to the invention are preferably selected from the group of skin diseases and skin-associated diseases or changes or conditions, such as, for example, psoriasis, skin ageing, skin wrinkling, skin rejuvenation, enlarged skin pores, cellulite, oily/greasy skin, folliculitis, actinic keratosis, precancerous actinic keratosis, skin lesions, sun-damaged and sun-stressed skin, crows' feet, skin ulcers, acne, acne rosacea, scars caused by acne, acne bacteria, photomodulation of greasy/oily sebaceous glands and their surrounding tissue, jaundice, jaundice of the newborn, vitiligo, skin cancer, skin tumours, Crigler-Najjar, dermatitis, atopic dermatitis, diabetic skin ulcers, and desensitisation of the skin.

Particular preference is given for the purposes of the invention to the treatment and/or prophylaxis of psoriasis, acne, cellulite, skin wrinkling, skin ageing, jaundice and vitiligo.

Further areas of application according to the invention for the devices are selected from the group of inflammatory diseases, rheumatoid arthritis, pain therapy, treatment of wounds, neurological diseases and conditions, oedema, Paget's disease, primary and metastasising tumours, connective-tissue diseases or changes in the collagen, fibroblasts and cell level originating from fibroblasts in tissues of mammals, irradiation of the retina, neovascular and hypertrophic diseases, allergic reactions, irradiation of the respiratory tract, sweating, ocular neovascular diseases, viral infections, particularly infections caused by herpes simplex or HPV (human papillomaviruses) for the treatment of warts and genital warts.

Particular preference is given for the purposes of the invention to the treatment and/or prophylaxis of rheumatoid arthritis, viral infections and pain.

Further areas of application according to the invention for the devices are selected from winter depression, sleeping sickness, irradiation for improving the mood, the reduction in pain particularly muscular pain caused by, for example, tension or joint pain, elimination of the stiffness of joints and the whitening of the teeth (bleaching).

Further areas of application according to the invention for the devices are selected from the group of disinfections. The devices can be used for the treatment of any type of objects (non-living materials) or subjects (living materials such as, for example, humans and animals) for the purposes of disinfection, sterilisation or preservation. This includes, for example, the disinfection of wounds, the reduction in bacteria, the disinfection of surgical instruments or other articles, the disinfection or preservation of foods, of liquids, in particular water, drinking water and other drinks, the disinfection of mucous membranes and gums and teeth. Disinfection here is taken to mean the reduction in the living microbiological causative agents of undesired effects, such as bacteria and germs.

The devices according to the invention emit, in particular, in the UV and blue region of the spectrum. The precise wavelength can be adjusted towards longer wavelengths without difficulties by the person skilled in the art depending on the respective application.

In a particularly preferred embodiment of the present invention, the device is an organic light-emitting diode (OLED) or an organic light-emitting electrochemical cell (OLEC) which are employed for the purposes of phototherapy. Both the OLED and the OLEC can have a planar or fibre-like structure having any desired cross section (for example round, oval, polygonal, square) with a single- or multilayered structure. These OLECs and/or OLEDs can be installed in other devices which comprise further mechanical, adhesive and/or electronic elements (for example battery and/or control unit for adjustment of the irradiation times, intensities and wavelengths). These devices comprising the OLECs and/or OLEDs according to the invention are preferably selected from the group comprising plasters, pads, tapes, bandages, sleeves, blankets, hoods, sleeping bags, textiles and stents.

The use of the said devices for the said therapeutic and/or cosmetic purpose is particularly advantageous compared with the prior art, since homogeneous irradiation in the high-energy blue region and/or in the UV region of lower irradiation intensities is possible at virtually any site and at any time of day with the aid of the devices according to the invention using the OLEDs and/or OLECs. The irradiation can be carried out as an inpatient, as an outpatient and/or by the patient themselves, i.e. without introduction and/or guidance by medical or cosmetic specialists. Thus, for example, plasters can be worn under clothing, so that irradiation is also possible during working hours, in leisure time or during sleep. Complex inpatient/outpatient treatments can in many cases be avoided or their frequency reduced. The devices according to the invention may be intended for re-use or be disposable articles, which can be disposed of after use once, twice or more times.

Further advantages over the prior art are, for example, lower evolution of heat and emotional aspects. Thus, newborn being treated owing to jaundice typically have to be irradiated blindfolded in an incubator without physical contact with the parents, which represents an emotional stress situation for parents and newborn. With the aid of a blanket according to the invention comprising the OLEDs and/or OLECs according to the invention, the emotional stress can be reduced significantly. In addition, better temperature control of the child is possible due to reduced heat production of the devices according to the invention compared with conventional irradiation equipment.

The present invention therefore also relates, in particular, to the device according to the invention for use in medicine for phototherapy.

The present invention also relates to the device according to the invention for use for the treatment of the skin by means of phototherapy.

The present invention also relates to the device according to the invention for use for the treatment of psoriasis by means of phototherapy.

The present invention also relates to the device according to the invention for use for the treatment of jaundice by means of phototherapy.

The present invention also relates to the device according to the invention for use for the treatment of jaundice of the newborn by means of phototherapy.

The present invention also relates to the device according to the invention for use for the treatment of acne by means of phototherapy.

The present invention also relates to the device according to the invention for use for the treatment of inflammation by means of phototherapy.

The present invention also relates to the device according to the invention for use for the treatment of atopic eczema by means of phototherapy.

The present invention also relates to the device according to the invention for use for the treatment of skin ageing by means of phototherapy

The present invention furthermore relates to the use of the devices according to the invention in the cosmetics area for phototherapy.

In particular, the present invention relates to the use of the devices according to the invention for the phototherapeutic reduction and/or for the phototherapeutic prevention of the formation of skin wrinkles and skin ageing.

The present invention also relates to a method for the treatment of the skin by phototherapy using a device according to the invention.

For the purposes of the present invention, a straight-chain or branched or cyclic alkyl group or an aliphatic hydrocarbon radical is taken to mean an alkyl, alkenyl and alkynyl groups preferably having 1 or 3 to 40 C atoms respectively, more preferably 1 or 3 to 20 C atoms respectively, even more preferably 1 or 3 to 10 C atoms respectively and most preferably 1 or 3 to 6 C atoms respectively. Cyclic alkyl groups can be mono-, bi- or polycyclic alkyl groups. Individual —CH— or —CH₂ groups may be replaced by N, NH, O or S. Examples of alkyl groups are the radicals methyl, ethyl, n-propyl, i-propyl, n-butyl, i-butyl, s-butyl, t-butyl, 2-methylbutyl, n-pentyl, s-pentyl, t-pentyl, cyclopentyl, n-hexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, heptynyl or octynyl.

An alkoxy group or thioalkyl group is taken to mean an alkyl group as defined above which is bonded via an oxygen atom or a sulfur atom. Preferred alkoxy groups are methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy and 2,2,2-trifluoroethoxy. Preferred thioalkyl groups are methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethyl-thio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio.

In general, alkyl, alkoxy or thioalkyl groups or aliphatic hydrocarbon radicals in accordance with the present invention can be straight-chain, branched or cyclic, where one or more non-adjacent CH₂ groups may be replaced by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵; furthermore, one or more H atoms may also be replaced by D, F, Cl, Br, I, CN or NO₂, preferably F, Cl or CN, further preferably F or CN, particularly preferably CN.

The mono- or polycyclic aliphatic ring system can be a ring system which only consists of CH₂ units, but one or more of the CH₂ groups may also be replaced by O, S or NH.

The term “aryl” or “heteroaryl” in connection with the terms “aryloxy”, “aryl-alkoxy”, heteroaryloxy” or “arylalkyl” is taken to mean an aromatic or heteroaromatic hydrocarbon radical, which may be mono- or polycyclic and preferably contains 6 or 5 to 60, more preferably 6 to 20, most preferably 5 or 6 aromatic ring atoms. If the unit is an aromatic unit, it preferably contains 6 to 20, more preferably 6 to 10, most preferably 6 carbon atoms as ring atoms. If the unit is a heteroaromatic unit, at least one of the ring atoms is a heteroatom. The heteroatoms are preferably selected from N, O and/or S. An aromatic or heteroaromatic unit here is taken to mean either a simple aromatic ring, i.e. benzene, or a simple heteroaromatic ring, for example pyridine, pyrimidine, thiophene, etc., or a condensed aryl or heteroaryl group, for example naphthalene, anthracene, phenanthrene, quinoline, isoquinoline, benzothiophene, benzofuran and indole etc.

Examples according to the invention of the aromatic or heteroaromatic hydrocarbon radical are accordingly: benzene, naphthalene, anthracene, phenanthrene, pyrene, chrysene, benzanthracene, perylene, naphthacene, pentacene, benzopyrene, furan, benzofuran, isobenzofuran, dibenzofuran, thiophene, benzothiophene, isobenzothiophene, dibenzothiophene, pyrrole, indole, isoindole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubin, naphthyridine, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

The term “alkyl” in connection with the term “arylalkyl” is taken to mean an alkyl group as defined above which is bonded to an aryl group.

The terms “aryloxy”, “arylalkoxy” or “heteroaryloxy” are taken to mean the groups “aryl”, “arylalkyl” or “heteroaryl” which are bonded via an oxygen atom.

An aromatic ring system having 6 to 18 ring atoms, preferably 6 to 15 ring atoms and even more preferably 6 to 10 ring atoms is taken to mean a system which contains no aromatic heteroatoms. This system is not necessarily taken to mean only one which contains aromatic groups, but also one in which, in addition, a plurality of aromatic groups may be interrupted by a short non-aromatic unit (<10% of the atoms other than H, preferably <5% of the atoms other than H), such as, for example, sp³-hybridised C, O, N, etc. These aromatic ring systems may be monocyclic or polycyclic, i.e. they may have one ring (for example phenyl) or two or more rings, which may also be condensed (for example naphthyl) or covalently linked (example biphenyl), or contain a combination of condensed and linked rings.

Preferred aromatic ring systems are, for example, phenyl, biphenyl, triphenyl, naphthyl, anthracyl, phenanthryl, dihydrophenanthryl, pyrene, dihydropyrene, chrysene, tetracene, fluorene and indole.

A heteroaromatic ring system in the sense of this invention is preferably taken to mean a heteroaromatic ring system having 5 to 18 ring atoms, preferably 5 to 14, particularly preferably 5 to 10 ring atoms. The heteroaromatic ring system contains at least one heteroatom selected from N, O and S (remaining atoms are carbon). A heteroaromatic ring system is, in addition, intended to be taken to mean a system which does not necessarily contain only aromatic or heteroaromatic groups, but also in which, in addition, a plurality of aromatic or heteroaromatic groups may be interrupted by a short non-aromatic unit (<10% of the atoms other than H, preferably <5% of the atoms other than H), such as, for example, sp³-hybridised C, O, N, etc. These heteroaromatic ring systems may be monocyclic or polycyclic, i.e. they may have one ring (for example pyridyl) or two or more rings, which may also be condensed or covalently linked, or contain a combination of condensed and linked rings

Preferred heteroaromatic ring systems are, for example, 5-membered rings, such as pyrrole, pyrazole, imidazole, 1,2,3-triazole, 1,2,4-triazole, tetrazole, furan, thiophene, selenophene, oxazole, isoxazole, 1,2-thiazole, 1,3-thiazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 6-membered rings, such as pyridine, pyridazine, pyrimidine, pyrazine, 1,3,5-triazine, 1,2,4-triazine, 1,2,3-triazine, 1,2,4,5-tetrazine, 1,2,3,4-tetrazine, 1,2,3,5-tetrazine, or condensed groups, such as indole, isoindole, indolizine, indazole, benzimidazole, benzotriazole, purine, naphthimidazole, phenanthrimidazole, pyridimidazole, pyrazinimidazole, quinoxalinimidazole, benzoxazole, naphthoxazole, anthroxazole, phenanthroxazole, isoxazole, benzothiazole, benzofuran, isobenzofuran, dibenzofuran, quinoline, isoquinoline, pteridine, benzo-5,6-quinoline, benzo-6,7-quinoline, benzo-7,8-quinoline, benzoisoquinoline, acridine, phenothiazine, phenoxazine, benzopyridazine, benzopyrimidine, quinoxaline, phenazine, naphthyridine, azacarbazole, benzocarboline, phenanthridine, phenanthroline, thieno[2,3b]thiophene, thieno[3,2b]thiophene, dithienothiophene, isobenzothiophene, dibenzothiophene, benzothiadiazo-thiophene or combinations of these groups. Particular preference is given to imidazole, benzimidazole and pyridine.

The devices, compositions and formulations according to the invention are distinguished by the following surprising advantages over the prior art:
1. The devices according to the invention emit in the UV-A and UV-B region.
2. The emitter compounds required for the preferred emission are readily accessible.
3. The use of mixed hosts enables the operating voltage to be reduced and the radiation intensity to be increased.
4. The use of blocking layers enables the operating voltage to be significantly reduced and the radiation intensity to be increased.
5. The devices according to the invention can easily be processed from solution.

These above-mentioned advantages are not accompanied by an impairment in the other electronic properties.

It should be pointed out that variations of the embodiments described in the present invention fall within the scope of this invention. Each feature disclosed in the present invention can, unless explicitly excluded, be replaced by alternative features which serve the same, an equivalent or a similar purpose. Thus, each feature disclosed in the present invention should, unless stated otherwise, be regarded as an example of a generic series or as an equivalent or similar feature.

All features of the present invention can be combined with one another in any way, unless certain features and/or steps are mutually exclusive. This applies, in particular, to preferred features of the present invention. Equally, features of non-essential combinations can be used separately (and not in combination).

It should furthermore be pointed out that many of the features, and in particular those of the preferred embodiments of the present invention, should be regarded as inventive themselves and not merely as part of the embodiments of the present invention. Independent protection may be granted for these features in addition or as an alternative to each invention claimed at present.

The teaching regarding technical action disclosed with the present invention can be abstracted and combined with other examples.

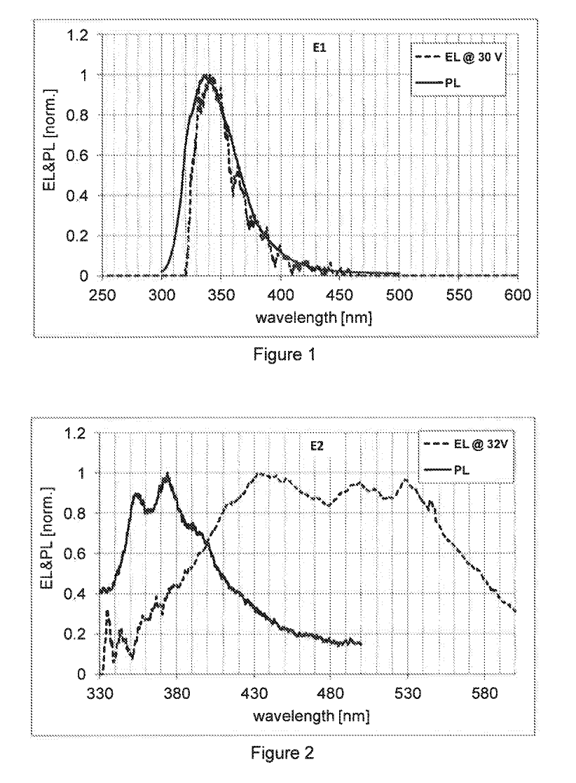

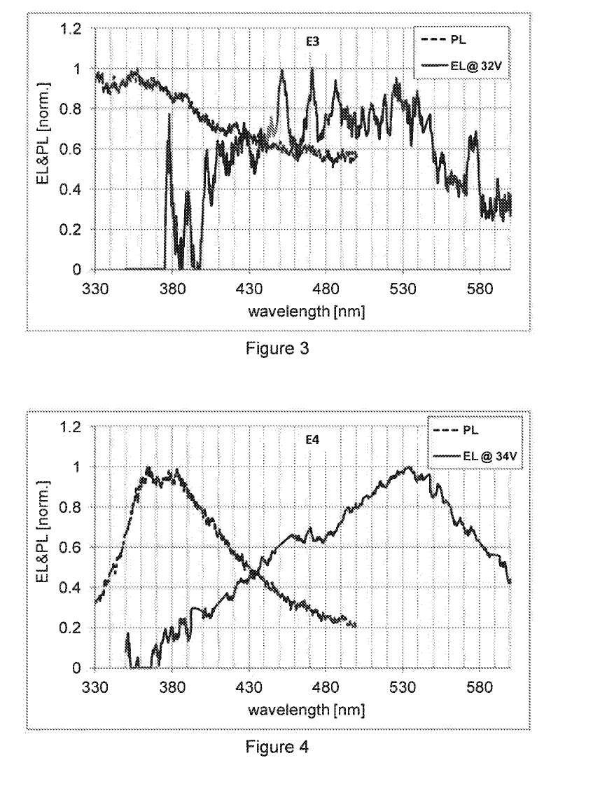

The invention is explained in greater detail by the following examples and FIGS. 1 to 4 without wishing to restrict it thereby.

### BRIEF DESCRIPTION OF THE FIGURES

FIGS. 1 to 4, 10 and 11 show the electroluminescence (EL) and photoluminescence spectra (PL) of devices according to the invention comprising emitters E1 to E4, E11 and E12.

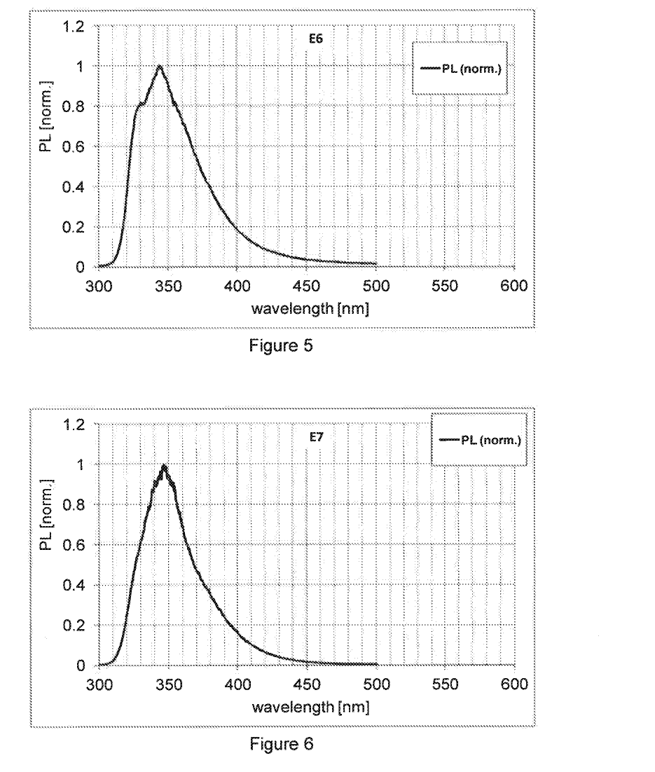

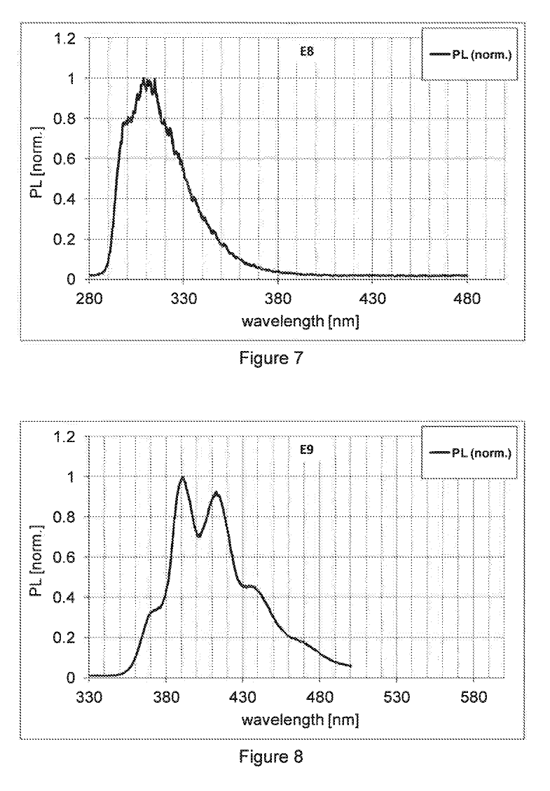

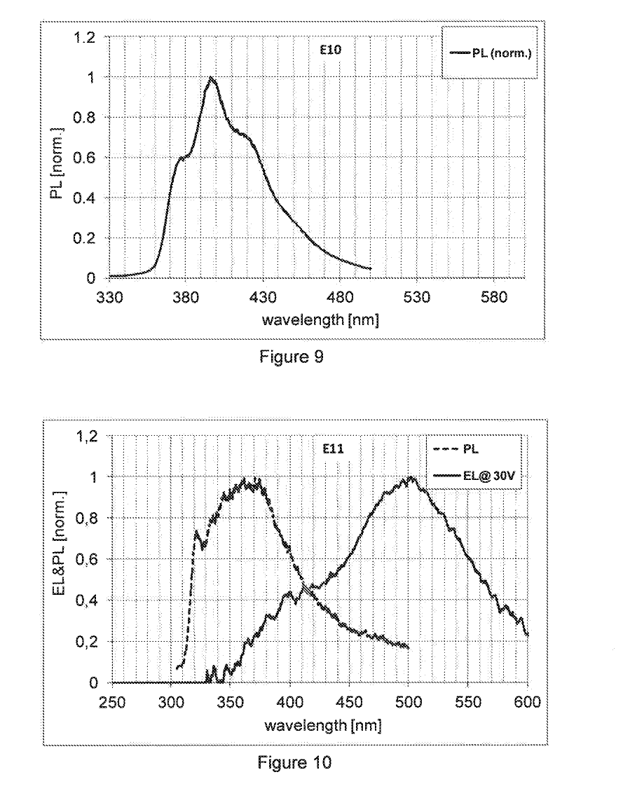

FIGS. 5 to 9, 12 and 13 show the photoluminescence spectra of devices according to the invention comprising emitters E6 to E10, E13 and E14.

FIG. 14 shows the combined phospholuminescence spectrum of devices according to the invention comprising emitters E2 to E4.

### EXAMPLES

### Example 1

### Materials

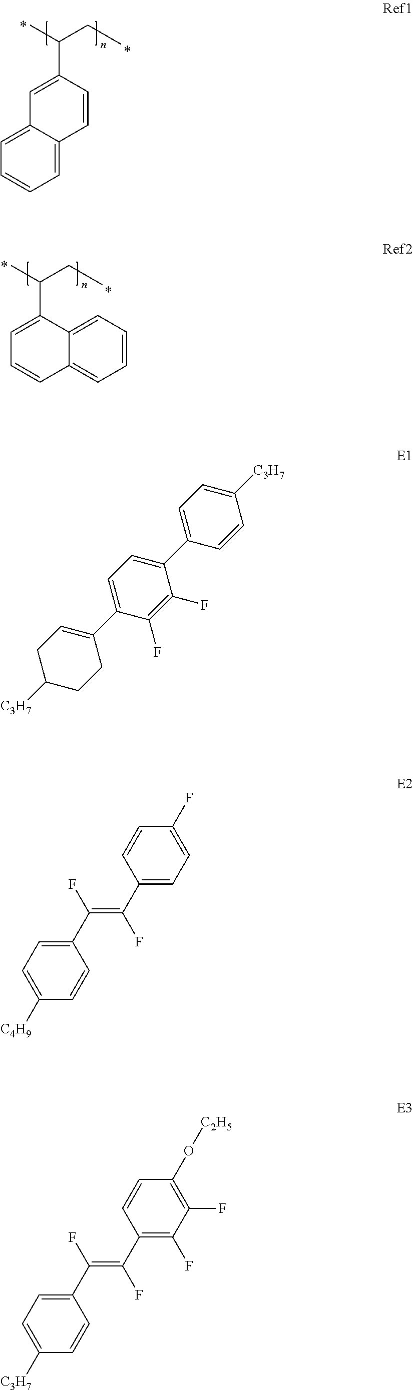

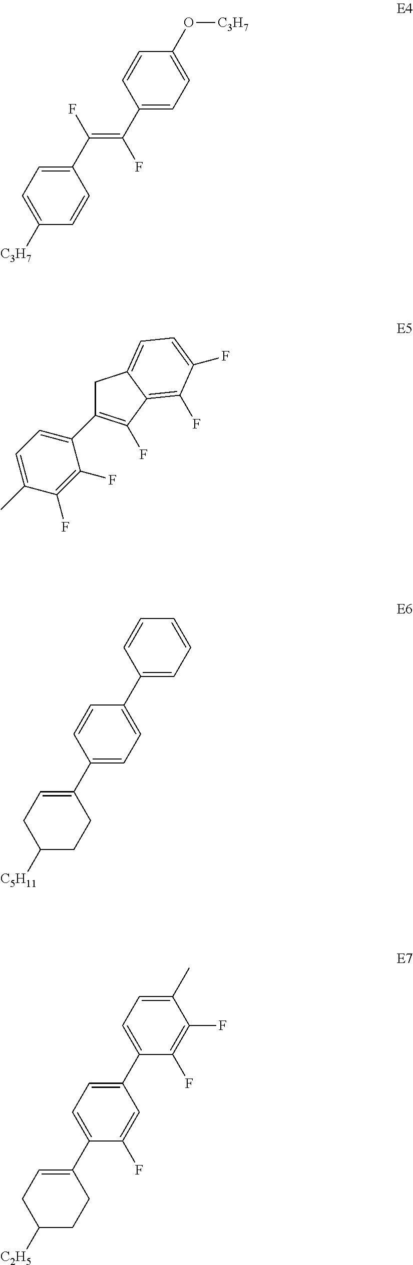

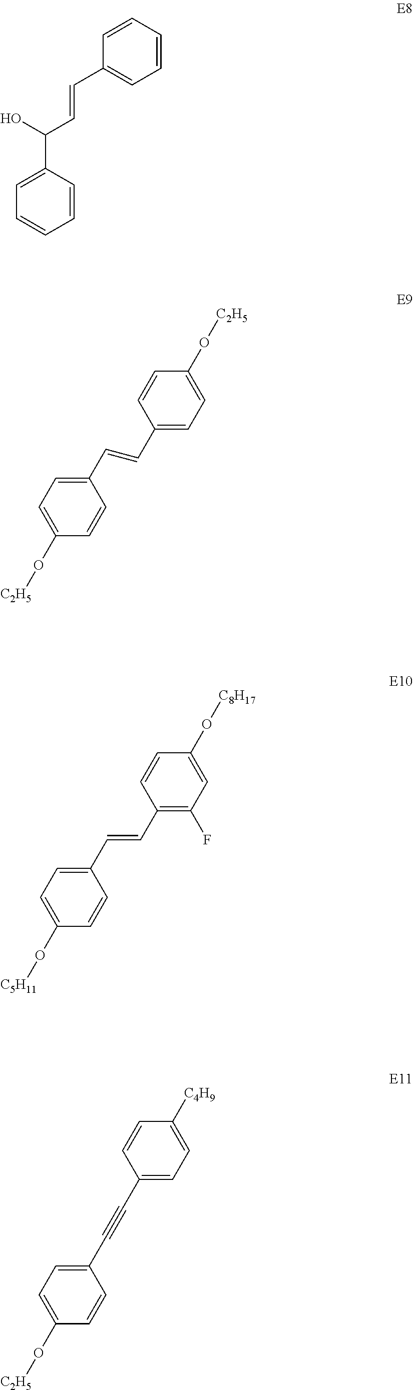

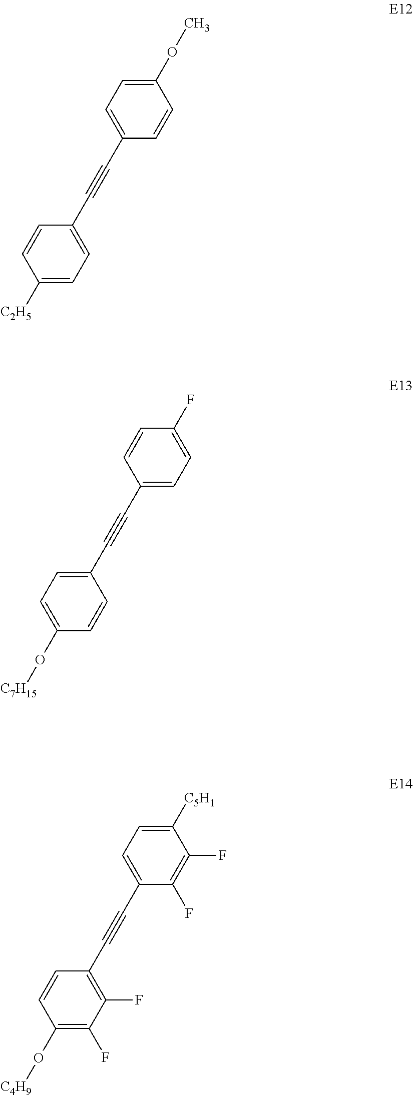

E1 to E10 are compounds of the formula (1). E11 to E14 are compounds of the formula (2). Ref1 (poly(2-vinylnaphthalene)) and Ref2 (poly(1-vinylnaphthalene)) are reference compounds and can be purchased from Sigma-Aldrich. Table 1 shows CAS numbers and other references for the compounds and the preparation thereof.

In addition, polystyrene (PS) from Fluka having a molecular weight Mw of 200 kdaltons is used as host.

TABLE 1

| Compound | CAS No. | Reference |
|---|---|---|
| E1 | | M. Hird et al., J. Materials Chemistry |
| | | 1995, 5(12), 2239-2245 |
| E2 | 193755-15-6 | JP11302651 A |
| E3 | 1029519-55-8 | DE102007057679 A1 |
| E4 | 1031801-25-8 | DE102007057679 A1 |
| E6 | 312747-96-9 | CN 101811985 |
| E7 | 1003218-28-7 | WO2008009417 A1 |
| E8 | 62668-02-4 | |
| E9 | 35135-54-7 | US 2346048 |
| E11 | 85583-83-1 | JP09255954 A |
| E12 | 63221-88-5 | WO9921816 A1 |
| E13 | 134143-76-3 | DE4105742 A1 |
| E14 | 197012-86-5 | WO9921816 A1 |

### Synthesis of Compound E5

### a) Synthesis of 2-(2,3-difluoro-4-methylphenyl)ethanol

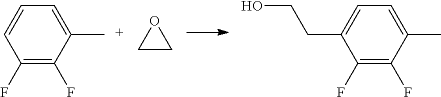

2,3-Difluorotoluene (256.246 g, 2 mol; CAS 3828-49-7) are cooled to −70° C. under nitrogen. At constant temperature of −65° C. to −70° C., 1375 ml (2.19 mol) of butyllithium (15% solution in n-hexane) are added dropwise, and, after a further 30 minutes, the liquefied ethylene oxide (88.1 g, 2 mol) is added in one portion. The reaction is stirred at low temperature for 30 minutes. The boron trifluoride/diethyl ether complex (251.204 ml, 2 mol) is then slowly added dropwise at constant temperature. The reaction is warmed to room temperature over hours, poured onto ice-water, acidified using hydrochloric acid and extracted with MtB ether. The virtually colourless oil is filtered through silica gel (hexane/MtB 3:2) and evaporated to dryness (406 g, purity 78%, yield 92%, virtually colourless oil)

### b) Synthesis of 2,3-difluoro-4-methylphenyl)acetic acid

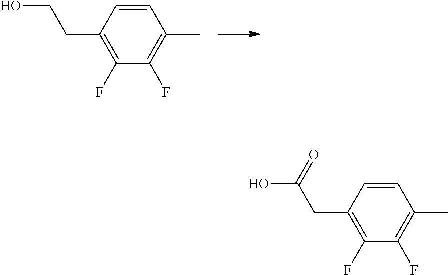

2-(2,3-Difluoro-4-methylphenyl)ethanol (10 g, 58.08 mmol) are dissolved in acetone 150 ml), 95-97% sulfuric acid (4 ml, 72 mmol) and Celite (15 g) are added. With ice-cooling, the Jones reagent (25.28 ml) is added dropwise at 30° C. The reaction mixture is brought to room temperature and filtered off with suction. The filtrate evaporated to dryness is stirred with water and MtB ether, extracted with aqueous slightly alkaline NaHCO3 solution. The residue is recrystallised from 146 ml of heptane (4.5 g, brown crystals, yield 50%).

### c) Synthesis of (2,3-difluoro-4-methylphenyl) acetyl chloride

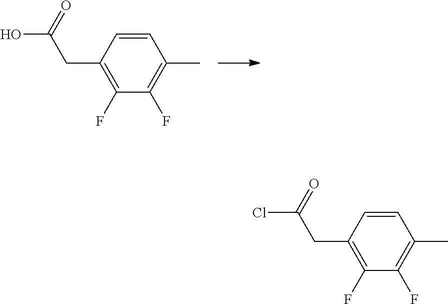

2,3-Difluoro-4-methylphenyl)acetic acid (115.4 g, 620 mmol) dissolved in dichloromethane (275 ml) and N,N-dimethylformamide (1 ml) are warmed to 30° C., and thionyl chloride (71.3 ml, 982.8 mmol) is added. The reaction is stirred further at 40° C. until the evolution of gas is complete. The reaction is distilled to dryness (117.2 g of violet oil, yield 92%).

### d) Synthesis of 2-(2,3-difluoro-4-methylphenyl)-1-(2,3-difluorophenyl)ethane

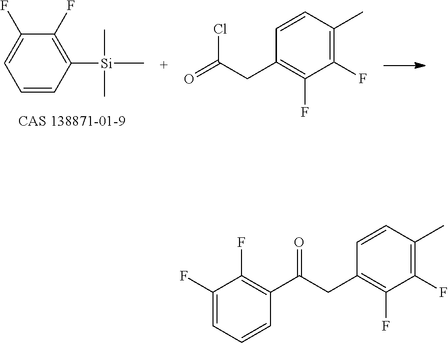 [SMILES: C[Si](C)(C)c1cccc(F)c1F ; Cc1ccc(CC(=O)Cl)c(F)c1F ; Cc1ccc(CC(=O)c2cccc(F)c2F)c(F)c1F]

(2,3-Difluoro-4-methylphenyl) acetyl chloride (114.573 g, 560 mmol) are dissolved in cyclohexane (400 ml) under nitrogen. (2,3-Difluorophenyl)-trimethylsilane (105.503 g, 560 mmol) dissolved in 100 ml of cyclohexane are added dropwise. Aluminium chloride (82.157 g, 616 mmol) is introduced in portions. When the reaction is complete, the reaction mixture is poured onto 2 l of ice-water and with a mixture of tetrahydrofuran (500 ml) and MtB ether (500 ml). The residue is recrystallised from 800 ml of ethanol (104.5 g, colourless crystals, yield 65%).

### e) Synthesis of 2-(2,3-difluoro-4-methylphenyl)-1-(2,3-difluorophenyl)propenone

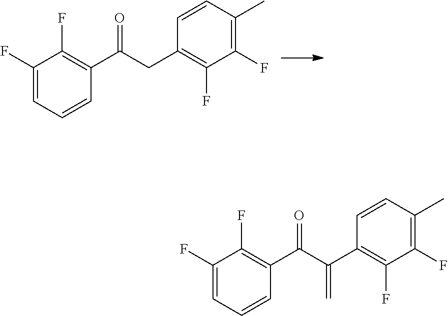 [SMILES: Cc1ccc(CC(=O)c2cccc(F)c2F)c(F)c1F ; C=C(C(=O)c1cccc(F)c1F)c1ccc(C)c(F)c1F]

2-(2,3-Difluoro-4-methylphenyl)-1-(2,3-difluorophenyl)ethane (4.94 g 17.5 mmol) are initially introduced in N,N,N′,N′-tetramethyldiaminomethane (25 ml). Acetic anhydride (25 ml, 264.5 mmol) is added dropwise with cooling. When the reaction is complete, the reaction mixture is poured onto ice-water and extracted with MtB ether, the organic phase is washed with aqueous NaHCO₃ solution and evaporated to dryness (4.9 g, yield 91%)

### f) Synthesis of 2-(2,3-difluoro-4-methylphenyl)-6,7-difluoroindan-1-one

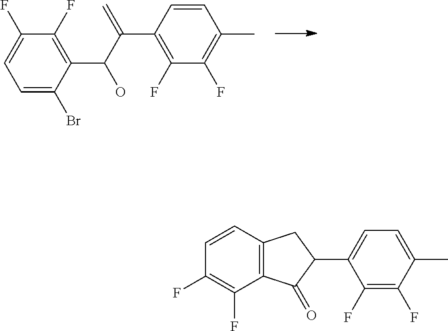 [SMILES: C=C(c1ccc(C)c(F)c1F)C(O)c1c(Br)ccc(F)c1F ; Cc1ccc(C2Cc3ccc(F)c(F)c3C2=O)c(F)c1F]

Trifluoromethanesulfonic acid (50 ml, 570 mmol) is initially introduced under N₂, cooled to about 2° C., and 2-(2,3-difluoro-4-methylphenyl)-1-(2,3-difluorophenyl)propenone (26.8 g, 82.7 mmol) dissolved in 25 ml of dichloromethane is added dropwise with ice-cooling at max. 10° C. The mixture is stirred at room temperature for 2 h. The reaction mixture is slowly poured onto a mixture of ice-water and MtB ether, the org. phase is shaken with sat. NaHCO₃ solution. The residue is chromatographed (toluene, SiO₂) (11.8 g of yellow crystals, yield 46%).

### g) Synthesis of Thioketal

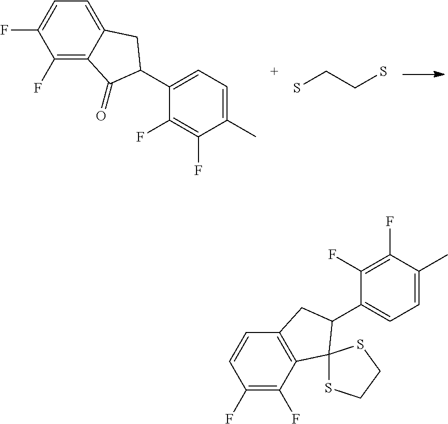

2-(2,3-Difluoro-4-methylphenyl)-6,7-difluoroindan-1-one (29.8 g, 101 mmol) and 1,2-ethanedithiol (17 ml, 202 mmol) are initially introduced in 300 ml of dichloromethane under nitrogen and cooled to 10° C. The boron trifluoride/ethyl ether complex (72.5 ml) is added dropwise at constant temperature. The reaction mixture is stirred at 5-10° C. for 1 h and subsequently warmed to room temperature. The reaction mixture is carefully poured into a 10% aqueous NaHCO₃ solution (1100 ml) and stirred for a further 1 h. The organic phase is evaporated to dryness. The crude product was recrystallised from 1200 ml of heptane and filtered off with suction at 5° C. (32.1 g of colourless crystals, yield 85%).

### h) Synthesis of synthesis of 2-(2,3-difluoro-4-methylphenyl)-3,4,5-trifluoro-1H-indene

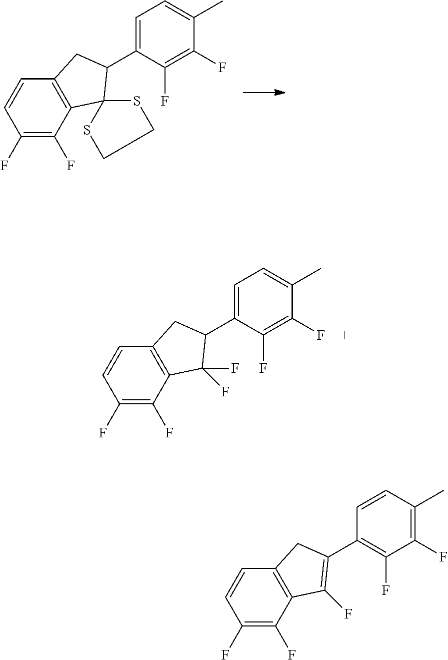 [SMILES: Cc1ccc(C2Cc3ccc(F)c(F)c3C23SCCS3)c(F)c1F ; Cc1ccc(C2Cc3ccc(F)c(F)c3C2(F)F)c(F)c1F ; Cc1ccc(C2=C(F)c3c(ccc(F)c3F)C2)c(F)c1F]

In a Teflon apparatus, N-iodosuccinimide (9.281 g, 41.25 mmol) is suspended in 60 ml of dichloromethane, the suspension is cooled to −70° C., and hydrogen fluoride 65% solution in pyridine (10.731 ml, 398 mmol) is added relatively rapidly at less than −35° C. The mixture is re-cooled to −70° C., and a solution of the thioketal (3.715 g, 10 mmol) in 20 ml of dichloromethane is added dropwise at less than −65° C. The mixture is stirred at low temperature for a further 4 h and subsequently warmed to room temperature. The reaction mixture is poured into a cold mixture of 600 ml of 1 N NaOH and 20 ml of 39% NaHSO₃ solution, extracted with dichloromethane, extract is shaken with 150 ml of 2 N HCl, organic phase is dried and evaporated. The crude product is taken up in hot heptane and filtered through a short silica-gel frit. The colourless filtrate is evaporated in a rotary evaporator, and the residue (310 mg of colourless crystals) is recrystallised from 30 ml of ethanol. (237 mg of colourless crystals, yield 7%).

### Synthesis of Compound E10

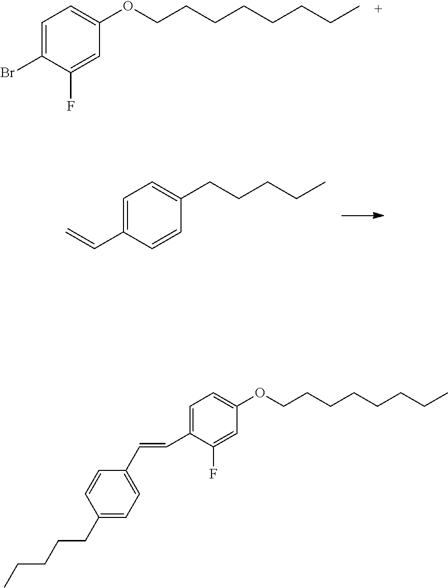

Palladium(II) acetate (0.025 eq), triethylamine (40 mmol) and tri-O-tolyl-phosphine (4 mmol) are added to a solution of 20 mmol (3.48 g) of 1-ethenyl-4-pentylbenzene and 20 mmol (6.06 g) of 1-bromo-2-fluoro-4-(octyloxy)benzene in 150 ml of acetonitrile and boiled reflux for 12 h. The reaction mixture is filtered and evaporated. The residue is filtered through silica gel (heptane/ethyl acetate) and evaporated (7.12 g, 18 mmol, yield 90%).

### Example 2

### Quantum-Chemical Calculations

The HOMO (highest occupied molecular orbital) and LUMO (lowest unoccupied molecular orbital) positions as well as the triplet/singlet level and oscillator strength of the organic compounds are determined via quantum-chemical calculations. To this end, the “Gaussian03W” program package (Gaussian Inc.) is used. In order to calculate organic substances without metals, firstly a geometry optimisation is carried out by means of the semi-empirical “Ground State/Semi-empirical/Default Spin/AM1” method (Charge 0/Spin Singlet). An energy calculation is subsequently carried out on the basis of the optimised geometry. In this, the “TD-SCF/DFT/Default Spin/B3PW91” method with the “6-31G(d)” base set (Charge 0/Spin Singlet) is used. The most important results are HOMO/LUMO levels, energies for the triplet and singlet (S₁) excited states and the oscillator strength (f). The first excited states (S₁ and T₁) are the most important here. S₁ stands for the first excited singlet level and T₁ stands for the first excited triplet level. The energy calculation gives the HOMO HEh or LUMO LEh in hartree units. The HOMO and LUMO values in electron volts (eV) are determined therefrom as follows, where these relationships arise from the calibration with reference to cyclic voltammetry measurements (CV):

HOMO(eV)=((HEh*27.212)−0.9899)/1.1206

LUMO(eV)=((LEh*27.212)−2.0041)/1.385

For the purposes of this application, these values are to be regarded as the energetic position of the HOMO level or LUMO level of the materials. As an example, an HOMO of −0.21634 hartrees and an LUMO of −0.03521 hartrees are obtained from the calculation for compound E1 (see also Table 1), which a calibrated HOMO of −6.14 eV, a calibrated LUMO of −2.14 eV.

It is known to the person skilled in the art that the quantum-chemical calculations, as described here, can be employed very well for the said purposes. The calculations give results which correlate very well with experimentally determined data.

TABLE 2
Energy levels of Ref1, Ref2, E1-E14

| Material | HOMO [eV] | LUMO [eV] | S1 [eV] | f |
|---|---|---|---|---|
| Ref1 | −6.06 | −2.19 | 4.31 | 0.08 |
| Ref2 | −6.08 | −2.13 | 4.24 | 0.03 |
| E1 | −6.14 | −2.14 | 4.19 | 0.77 |
| E2 | −5.97 | −2.42 | 4.06 | 0.97 |
| E3 | −6.04 | −2.46 | 4.06 | 1.04 |
| E4 | −5.68 | −2.23 | 4.00 | 1.13 |
| E5 | −6.07 | −2.71 | 3.86 | 0.77 |
| E6 | −5.91 | −2.09 | 4.18 | 0.76 |
| E7 | −6.09 | −2.25 | 4.20 | 0.69 |
| E8 | −6.14 | −2.01 | 4.33 | 0.40 |
| E9 | −5.40 | −2.14 | 3.79 | 1.19 |
| E10 | −5.62 | −2.33 | 3.89 | 1.11 |
| E11 | −5.63 | −2.20 | 4.03 | 1.34 |
| E12 | −5.67 | −2.23 | 4.04 | 1.24 |
| E13 | −6.41 | −2.06 | 4.43 | 0.00 |
| E14 | −6.15 | −2.60 | 4.03 | 1.34 |

### Example 3

### Solutions and Compositions

Solutions, as summarised in Table 3, are prepared as follows: firstly, the mixtures of host and emitter are dissolved in 10 ml of toluene and stirred until the solution is clear. The solution is filtered using a Millipore Millex LS, hydrophobic PTFE 5.0 μm filter.

TABLE 3
Compositions of the solutions

| | | Ratio (based on | |
|---|---|---|---|
| | Composition | weight) | Concentration |
| Solution Ref1 | Ref1 | 100% | 16 mg/ml |
| Solution Ref2 | Ref2 | 100% | 16 mg/ml |
| Solution x* | PS:Ex | 70%:30% | 16 mg/ml |

x is 1, 2, 3, . . . 14;
PS stands for polystyrene;

The solutions are used in order to coat the emitting layer of OLEDs. The corresponding solid composition can be obtained by evaporating the solvent from the solutions. This can be used for the preparation of further formulations.

### Example 4

### Production of the OLEDs

OLED Ref1, OLED Ref2, OLED1-OLED5 have the following structure: ITO/PEDOT/EML/cathode, where EML stands for the emission layer and ITO stands for the anode (indium tin oxide).

The OLEDs are produced using the corresponding solutions, as summarised in Table 2, in accordance with the following procedure:

1) Coating of 80 nm of PEDOT (Clevios™ P VP AI 4083) onto an ITO-coated glass substrate by spin coating; drying by heating at 180° C. for 10 min.

2) Coating of an 80 nm emitting layer by spin coating of one of the solution in accordance with Table 2.

3) Drying of the device by heating: 10 min at 180° C. for OLED Ref1 and OLEd Ref2; for OLED1-14 30 min at 50° C. and then 30 min in vacuo.

4) Vapour deposition of a Ba/Al cathode (3 nm/150 nm).

5) Encapsulation of the device.

### Example 5

### Characterisation of the OLEDs

Firstly, photoluminescence spectra (PL) of the emission layer (EML) and subsequently electroluminescence spectra (EL) of the OLEDs obtained in this way are measured, since the EL spectrum gives the most important indications of a functioning electroluminescent device.

The EL spectra are measured by means of Ocean Optics UBS2000.

The EL spectra of OLED1-4 are summarised in FIG. 1-4. OLED5 exhibits a weak EL spectrum, but with clear peaks at 367 nm and 388 nm. For the OLEDs with Ref1 and Ref2 as emitter, no EL spectra can be measured, even if the voltage is increased to 40 V. However, the OLEDs comprising emitters E1 to E5 according to the invention exhibit a clear EL spectrum having a significant proportion between 280 to 380 nm.

The PL spectra of E6-E10 were measured in an 80 nm layer of PS:Ex (30% by weight based on the entire layer) on quartz, and are shown in FIGS. 5-9. All these arrangements emit radiation in the UV region.

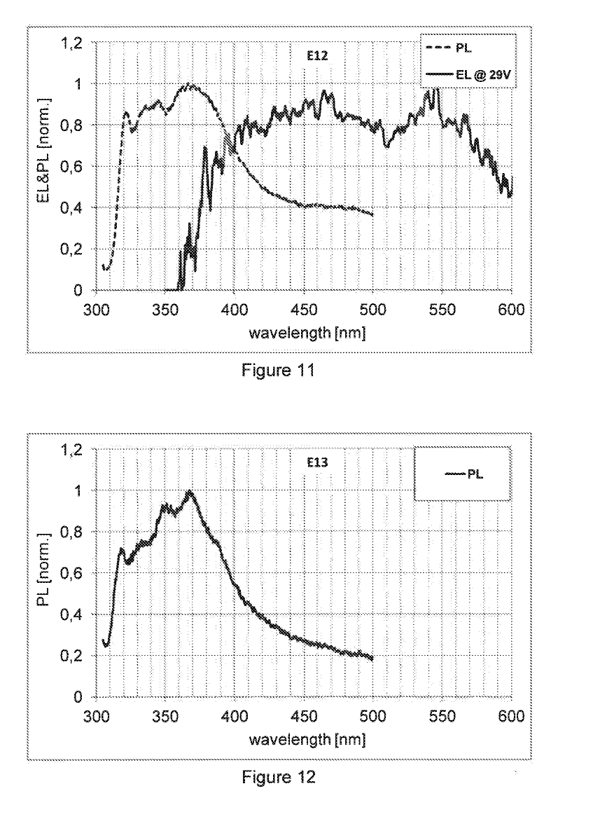

The EL spectra of OLED11-12 are summarised in FIGS. 10 and 11. However, the OLEDs comprising emitters E11 and E12 according to the invention exhibit a clear EL spectrum having a significant proportion between 280 to 380 nm.

OLED13 comprising E13 as emitter was not able to record an EL spectrum. The PL spectrum of E13 is shown in FIG. 12.

It is in fact very surprising that the emitters according to the invention emit in the UV region in an OLED having the simple layer structure indicated and comprising polystyrene as host. It is apparent that the person skilled in the art will be able to carry out further optimisation on the basis of the present invention and without inventive step.

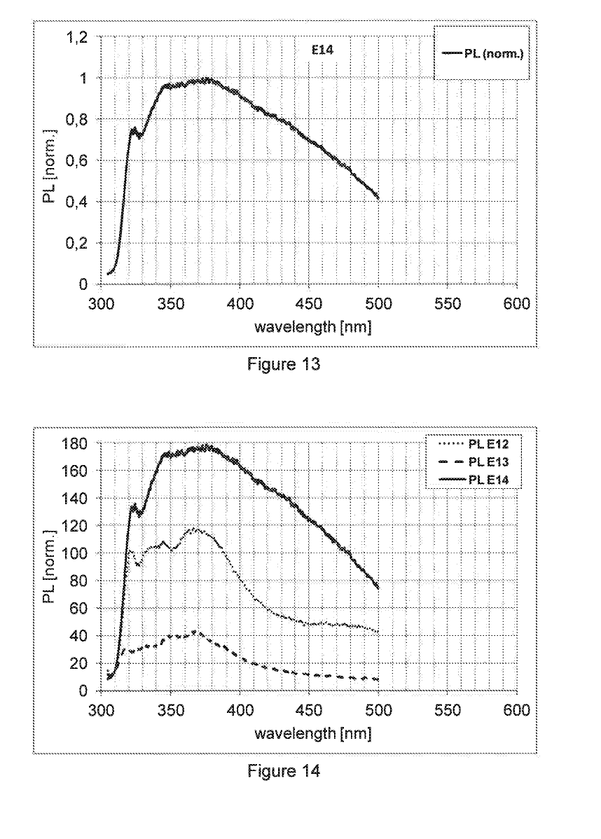

The PL spectra of E11-E14 were measured in an 80 nm layer of PS:Ex (30% by weight based on the layer) on quartz. For E14, only a PL spectrum was measured, which is shown in FIG. 13. The devices according to the invention all emit radiation in the UV region. The absolute PL intensity of E12-14 in an 80 nm thin layer was measured under the same conditions, i.e. the same excitation wavelength, the same gap width, the same integral times. The comparison is shown in FIG. 14. E13 shows only a very weak PL, which agrees with the oscillator strength f in Table 1.

1.-19. (canceled)

20. An electroluminescent device comprising at least two electrodes and at least one emitting layer between the electrodes, which comprises at least one compound of the following formula (1) or (2): 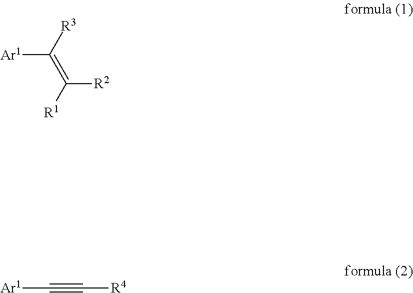
where the symbols used have the following meanings:
Ar¹ is an aromatic or heteroaromatic ring having 5 or 6 ring atoms, which is optionally substituted by one or more radicals R⁶, or a bicyclic condensed aromatic or heteroaromatic ring system having 10 to 12 ring atoms, which is optionally substituted by one or more radicals R⁶;
R¹, R², R³ and R⁴ are, independently of one another on each occurrence, H, D, F, Ar², N(R⁵)₂, CN, Si(R⁵)₃, B(OR⁵)₂, P(R⁵)₂, S(═O)R⁵, a straight-chain alkyl, alkoxy or thioalkoxy group having 1 to 40 C atoms or a branched or cyclic alkyl, alkoxy or thioalkoxy group having 3 to 40 C atoms, each of which is optionally substituted by one or more radicals R⁶, where one or more non-adjacent CH₂ groups is optionally replaced by R⁵C═CR⁵, Si(R⁵)₂, Ge(R³)₂, Sn(R⁵)₂, C═O, C═S, C═Se, C═NR⁵, P(═O)(R⁵), SO, SO₂, NR⁵, O, S or CONR⁵ and where one or more H atoms is optionally replaced by D, F, Cl, or CN, or an aryloxy, arylalkoxy or heteroaryloxy group having 5 to 60 aromatic ring atoms, which may in each case be substituted by one or more radicals R⁶, or a combination of two or more of these groups; where, in addition, two or more of the radicals R¹, R², R³ and R⁴ may form a mono- or polycyclic ring system, which is optionally substituted by one or more radicals R⁸, with one another; where, R¹ may also be a divalent group W, which is linked to Ar¹ of the formula (1), and where, if R² is equal to Ar², R³ may also be a divalent group W which is linked to Ar²;
Ar² has the same meaning as Ar¹;
W is, identically or differently on each occurrence, a non-aromatic bridge between the double or triple bond of the formula (1) or (2) and Ar¹ or Ar² and contains O, S, Se, N, Si, B, P and/or at least one group C(R⁷)₂;
R⁵ is, identically or differently on each occurrence, H, D, F, OH, N(R⁷)₂, CN, Si(R⁷)₃, B(OR⁷)₂, P(═O)(R⁷)₂, P(R⁷)₂, S(═O)R⁷, a straight-chain alkyl, alkoxy or thioalkoxy group having 1 to 40 C atoms or a branched or cyclic alkyl, alkoxy or thioalkoxy group having 3 to 40 C atoms, each of which is optionally substituted by one or more radicals R⁸, where one or more non-adjacent CH₂ groups is optionally replaced by R⁷C═CR⁷, C≡C, Si(R⁷)₂, Ge(R⁷)₂, Sn(R⁷)₂, C═O, C═S, C═Se, C═NR⁷, P(═O)(R⁷), SO, SO₂, NR⁷, O, S or CONR⁷ and where one or more H atoms is optionally replaced by D, F, Cl or CN, or an aromatic ring system having 6 to 18 aromatic ring atoms or a heteroaromatic ring system having 5 to 18 aromatic ring atoms, each of which is optionally substituted by one or more radicals R⁸, or a combination of two or more of these groups; two or more adjacent radicals R⁷ or R⁸ may form a mono- or polycyclic aliphatic ring system with one another here;
R⁶ has the same meaning as R⁵, but cannot be equal to H;
R⁷ is, identically or differently on each occurrence, H, D, F, a straight-chain alkyl, alkoxy or thioalkoxy group having 1 to 40 C atoms or a branched or cyclic alkyl, alkoxy or thioalkoxy group having 3 to 40 C atoms, an aromatic ring system having 6 to 18 aromatic ring atoms or a heteroaromatic ring system having 5 to 18 aromatic ring atoms, in which, in addition, one or more H atoms is optionally replaced by F; two or more substituents R⁷ may also form a mono- or polycyclic aliphatic ring system with one another here; and
R⁸ has the same meaning as R⁷, but cannot be equal to H;
with the proviso that the compound of the formula (1) or (2) does not contain any through-conjugated structure whose number of π electrons is more than 18.

21. The electroluminescent device according to claim 20, wherein Ar¹ or Ar² is, independently of one another on each occurrence, a compound of the following formula (3) or (4): 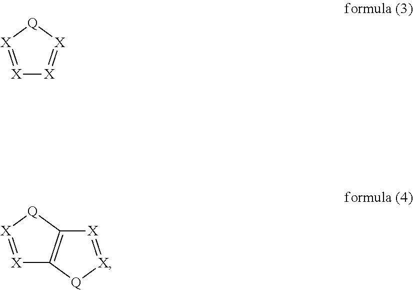
where Ar¹ or Ar² is optionally bonded to the double bond or triple bond of the formula (1) or (2) at any desired site of the compound of the formula (3) or (4); and
where the symbols used have the following meanings:
Q is on each occurrence, identically or differently, NR⁵, O, S or Se;
X is on each occurrence, identically or differently, CR⁵ or N;
R⁵ has the same meaning as defined in claim 20.

22. The electroluminescent device according to claim 20, wherein R¹ is equal to H, F or a group W which is linked to Ar¹.

23. The electroluminescent device according to claim 20, wherein R² is equal to Ar² or an arylalkyl group which is substituted by a radical R⁶ in the alkyl unit, or R², together with R³, forms a divalent unit —CH₂—(C(R⁷)₂)ₕ—CH₂—, where h is equal to 1, 2 or 3, and R⁷ has the same definition as defined in claim 20.

24. The electroluminescent device according to claim 20, wherein R³ is equal to H, F or a group W which is linked to Ar², or R², together with R³, forms a divalent unit —CH₂—(C(R⁷)₂)ₕ—CH₂—, where h is equal to 1, 2 or 3, and R⁷ has the same definition as defined in claim 20.

25. The electroluminescent device according to claim 20, wherein R⁴ is equal to Ar².

26. The electroluminescent device according to claim 20, wherein the compound of the formula (1) or (2) is a compound from the following formulae: 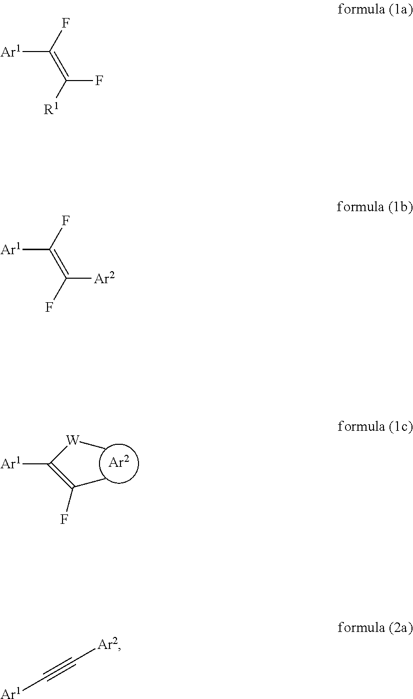
where the symbols used have the same meanings as in claim 20.

27. The electroluminescent device according to claim 20, wherein Ar¹ and/or Ar² is a phenyl group, which is optionally substituted by a radical R⁶.

28. The electroluminescent device according to claim 20, wherein R⁶ is equal to F, a straight-chain alkyl or alkoxy group having 1 to 20 C atoms or a branched or cyclic alkyl or alkoxy group having 3 to 20 C atoms, or an aromatic ring system having 6 to 10 aromatic ring atoms, each of which is optionally substituted by one or more radicals R⁸.

29. The electroluminescent device according to claim 20, wherein R⁸ is equal to F or a straight-chain alkyl or alkoxy group having 1 to 10 C atoms or a branched or cyclic alkyl or alkoxy group having 3 to 10 C atoms.

30. The electroluminescent device according to claim 20, wherein the compound of the formula (1) or (2) is employed as UV light-emitting compound.

31. The electroluminescent device according to claim 20, wherein the device comprises one or more additional layers between the electrodes.

32. The electroluminescent device according to claim 31, wherein the additional layer is selected from the group consisting of the following:
(a) an exciton-blocking layer which comprises an exciton-blocking material having a band gap of 3.6 eV or higher;
(b) an electron-blocking layer which comprises an electron-blocking material having an LUMO of higher than −2.2 eV; and
(c) a hole-blocking layer which comprises a hole-blocking material having an HOMO of lower than −6.0 eV.

33. The electroluminescent device according to claim 20, wherein the device emits radiation having a wavelength in the range from 280 nm to 380 nm.

34. The electroluminescent device according to claim 20, wherein the device is a device from the group consisting of the an organic light-emitting diode (OLED), a polymeric light-emitting diode (PLED), an organic light-emitting electrochemical cell (OLEC), organic light-emitting transistor (O-LET) and an organic light-emitting electrochemical transistor.

35. A composition comprising at least one of the compounds of the formula (1) or (2) as defined in claim 20 and at least one organic material or an organic semiconductor selected from the group consisting of an emitter, a host material, a matrix material, an electron-transport material (ETM), an electron-injection material (EIM), a hole-transport materials (HTM), a hole-injection material (HIM), an electron-blocking material (EBM), a hole-blocking material (HBM) and an exciton-blocking material (ExBM).

36. A formulation comprising the composition according to claim 35 and at least one solvent.

37. A device according to claim 20 for use in medicine for phototherapy.

38. A method for the treatment of the skin by phototherapy using the device according to claim 20.
